SEQUENCE LISTING (83 sequences)

SEQ ID NO: 1 - DNA, 2400 bp, Artificial
  Synthetic
acgtattttgaaactcaagatcgcattcatgcgtcttcacctggaaggggtccatgtgcccctccttctggccaccatgcgaagccacactgacgtgcctctccctccctccaggaagcctacgtgatggccagcgtggacaacccccacgtgtgccgcctgctgggcatctgcctcacctccaccgtgcagctcatcatgcagctcatgcccttcggctgcctcctggactatgtccgggaacacaaagacaatattggctcccagtacctgctcaactggtgtgtgcagatcgcaaaggtaatcagggaagggagatacggggaggggagataaggagccaggatcctcacatgcggtctgcgctcctgggatagcaagagtttgccatggggatatggaggctcagagcctggcatgaacatgaccctgaattcggatgcagagcttcttcccatgatgatctgtccctcacagcagggtcttctctgtttcagggcatgaactacttggaggaccgtcgcttggtgcaccgcgacctggcagccaggaacgtactggtgaaaacaccgcagcatgtcaagatcacagattttgggcgggccaaactgctgggtgcggaagagaaagaataccatgcagaaggaggcaaagtaaggaggtggctttaggtcagccagcattttcctgacaccagggaccaggctgccttcccactagctgtattgtttaacacatgcaggggaggatgctctccagacattctgggtgagctcgcagcagctgctgctggcagctggtctatcttccctagtgtggtaacctcatttccccataaagattcagaacacaaagatcatcctttcagagaaaataatgctcctagtacctgtagaggttaatatccgcaaatgacttgctattattgatggcaaatacacagaggaagccttcgcctgtcctcatgtattggtctctcatggcactgtactcttcttttccagctgtatccagtatgtccaacaaacaggtttcaccatctataaccacttgttttctgtaagaatcctgggggtgtggagggtaagggggcagggagggagggaagttcaatttttattaaaaaccacagggaatgcaatgctattgccaaggttaaataagcatctaactattcaagcccatttctgcctatctggtgctcggtgaggatcttcatgaggtagtcagtcaggtcccggccagccaggtccagacgcaggatggcatgggggagggcatacccctcgtagatgggcacagtgtgggtgaccccgtcaccggagtccatcacgatgccagtggtacggccagaggcgtacagggatagcacagcctggatagcaacgtacatggctggggtgttgaaggtctcaaacatgatctgtaaggcagagatacaccatgtcacactggggaagccactggggacagccaggccagacgggggacatgcagaaagtgcaaagaacacggctaagtgtgctggggtcttgggatggggagtctgttcagacctactgtgcacctacttaatacacactccaaggccgctttacacctgagatcacaccactgcactccagcttagaagatagagtgagactctgcctaaaaaaaaataaaaaataaaaaaataaaaatgaaaaaaatgcataacaacaaagaatatgaatatggatcacatctctaccagagttaatcaactgatgcaaactcttgcacaaatgctgaaagctgtaccatacctgtctggtcttggttgaggtttcaatgaatggaatcccgtaactcttggccagttcgtgggcttgttttgtatcaactgtccttgttggcaaatcacacttgtttcccactagcaccataggtacatcatccgagtcttttactcgcttaatctgctccctaaaaacgggaatatattatcagaacataagaaaaacaagattaggctgggttatatgcatggcattagcaaagactcaaaaaataaaaactataattactccttaatgtcagcttattatattcaatttaaacccacctataatggtgaatatcttcaaatgatttagtattatttatggcaaatacacaaagaaagccctccccagtcctcatgtactggtccctcattgcactgtactcctcttgacctgttgtgtcgagaatatccaagagacaggtttctccatcaattactacttgcttcctgtaggaatcctgagaagggagaaacacagtctggattattacagtgcaccttttacttcaaaaaaggtgttatatacaactcaacaacaaaaaattcaatttaaaaatgggcaaaggacttgaaaagacattgttcctgctcca

SEQ ID NO: 2 - DNA, 4800 bp, Artificial
  Synthetic
acttcacagccctgcgtaaacgtccctgtgctaggtcttttgcaggcacagcttttcctccatgagtacgtattttgaaactcaagatcgcattcatgcgtcttcacctggaaggggtccatgtgcccctccttctggccaccatgcgaagccacactgacgtgcctctccctccctccaggaagcctacgtgatggccatcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaaggtaatcagggaagggagatacggggaggggagaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacgtcatggtggctggacaacaaaaatggattggatcttccacacaattaaacagcatgcattgaactgaaaagataactgagaaaatgaaagctcactctggattccacactgcactgttaataactctcagcaggcaaagaccgattgcataggaattgcacaatccatgaacagcattagaatttacagcaagaacagaaatatgtatacccaaaataactttttactttctctcctcttattcctttaatacagaatatgggtaaagatgatccgacaagtgagagacaggatcaggtcagcgggctaccactgggcctcacctctatggtgggatcatattcatctacaaagtggttctggattagctggattgtcagtgcgcttttcccaacaccatctgctccaaccaccaccagtttgtactcagtcatttcacaccagcaagaacctgttggaaaccagtaatcagggttaattggcgagccacatctacagtactttaaagctttctataatcaatggaaatgaaaaccctagtgtgaccttccatttggttcttaaagtgactagagaacgcaaaaacaccggattaatatcggagcaatgtaaaatttattgaaaatgtatttgctttttctgtaaatcatctgtgaatccagaggggaaaaatatgacaaagaaagctatataagatattattttattttacagagtaacagactagctagagacaatgaattaagggaaaatgacaaagaacagctcaaagcaatttctacacgagatcctctctctaaaatcactgagcaggagaaagattttctatggagtcacaggtaagtgctaaaatggagattctctgtttctttttctttattacagaaaaaataactgaatttggctgatctcagcatgtttttaccatacctattggaataaataaagcagaatttacatgatttttaaactataaacattgcctttttaaaaacaatggttatcttccctagtgtggtaacctcatttccccataaagattcagaacacaaagatcatcctttcagagaaaataatgctcctagtacctgtagaggttaatatccgcaaatgacttgctattattgatggcaaatacacagaggaagccttcgcctgtcctcatgtattggtctctcatggcactgtactcttcttgtccagttgtatccagtatgtccaacaaacaggtttcaccatctataaccacttgttttctgtaagaatcctgggggtgtggagggtaagggggcagggagggagggaagttcaatttttattaaaaaccacagggaatgcaatgctattgccaaggttaaataagcatctaactattcaagcccatttctgcctatctggtttgagagcctggcatgaacatgaccctgaattcggatgcagagcttcttcccatgatgatctgtccctcacagcagggtcttctctgtttcagggcatgaactacttggaggaccgtcgcttggtgcaccgcgacctggcagccaggaacgtactggtgaaaacaccgcagcatgtcaagatcacagattttgggctggccaaacagctgggtgcggaagagaaagaataccatgcagaaggaggcaaagtaaggaggtggctttaggtcagccagcattttcctgacaccagggaccaggctgccttcccactagctgtattgtttaacacatgcaggggaggatgctctccagacattctgggtgagctcgcagcagctgctgctggcagctgggtccagcacttcacagccctgcgtaaacgtccctgtgctaggtcttttgcaggcacagcttttcctccatgagtacgtattttgaaactcaagatcgcattcatgcgtcttcacctggaaggggtccatgtgcccctccttctggccaccatgcgaagccacactgacgtgcctctccctccctccaggaagcctacgtgatggccatcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaaggtaatcagggaagggagatacggggaggggagaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacgtcatggtggctggacaacaaaaatggattggatcttccacacaattaaacagcatgcattgaactgaaaagataactgagaaaatgaaagctcactctggattccacactgcactgttaataactctcagcaggcaaagaccgattgcataggaattgcacaatccatgaacagcattagaatttacagcaagaacagaaatatgtatacccaaaataactttttactttctctcctcttattcctttaatacagaatatgggtaaagatgatccgacaagtgagagacaggatcaggtcagcgggctaccactgggcctcacctctatggtgggatcatattcatctacaaagtggttctggattagctggattgtcagtgcgcttttcccaacaccatctgctccaaccaccaccagtttgtactcagtcatttcacaccagcaagaacctgttggaaaccagtaatcagggttaattggcgagccacatctacagtactttaaagctttctataatcaatggaaatgaaaaccctagtgtgaccttccatttggttcttaaagtgactagagaacgcaaaaacaccggattaatatcggagcaatgtaaaatttattgaaaatgtatttgctttttctgtaaatcatctgtgaatccagaggggaaaaatatgacaaagaaagctatataagatattattttattttacagagtaacagactagctagagacaatgaattaagggaaaatgacaaagaacagctcaaagcaatttctacacgagatcctctctctaaaatcactgagcaggagaaagattttctatggagtcacaggtaagtgctaaaatggagattctctgtttctttttctttattacagaaaaaataactgaatttggctgatctcagcatgtttttaccatacctattggaataaataaagcagaatttacatgatttttaaactataaacattgcctttttaaaaacaatggttatcttccctagtgtggtaacctcatttccccataaagattcagaacacaaagatcatcctttcagagaaaataatgctcctagtacctgtagaggttaatatccgcaaatgacttgctattattgatggcaaatacacagaggaagccttcgcctgtcctcatgtattggtctctcatggcactgtactcttcttgtccagttgtatccagtatgtccaacaaacaggtttcaccatctataaccacttgttttctgtaagaatcctgggggtgtggagggtaagggggcagggagggagggaagttcaatttttattaaaaaccacagggaatgcaatgctattgccaaggttaaataagcatctaactattcaagcccatttctgcctatctggtttgagagcctggcatgaacatgaccctgaattcggatgcagagcttcttcccatgatgatctgtccctcacagcagggtcttctctgtttcagggcatgaactacttggaggaccgtcgcttggtgcaccgcgacctggcagccaggaacgtactggtgaaaacaccgcagcatgtcaagatcacagattttgggctggccaaacagctgggtgcggaagagaaagaataccatgcagaaggaggcaaagtaaggaggtggctttaggtcagccagcattttcctgacaccagggaccaggctgccttcccactagctgtattgtttaacacatgcaggggaggatgctctccagacattctgggtgagctcgcagcagctgctgctggcagctgggtccagc

SEQ ID NO: 3 - DNA, 2400 bp, Artificial
  Synthetic
agccctgcgtaaacgtccctgtgctaggtcttttgcaggcacagcttttcctccatgagtacgtattttgaaactcaagatcgcattcatgcgtcttcacctggaaggggtccatgtgcccctccttctggccaccatgcgaagccacactgacgtgcctctccctccctccaggaagcctacgtgatggccagcgccagcgtggtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaaggtaatcagggaagggagatacggggaggggaatatgcatggcattagcaaagactcaaaaaataaaaactataattactccttaatgtcagcttattatattcaatttaaacccacctataatggtgaatatcttcaaatgatttagtattatttatggcaaatacacaaagaaagccctccccagtcctcatgtactggtccctcattgcactgtactcctcgtgacctgctgtgtcgagaatatccaagagacaggtttctccatcaattactacttgcttcctgtaggaatcctgagaagggagaaacacagtctggattattacagtgcaccttttacttcaaaaaaggtgttatatacaactcaacaacaaaaaattcaatttaaaaatgggcaaaggacttgaaaagacattgttcctgctccaagcccatgccgtggctgctggtccccctgctgggccatgtctggcactgctttccagcatggtgagggctgaggtgacccttgtctctgtgttcttgtcccccccagcttgtggagcctcttacacccagtggagaagctcccaaccaagctctcttgaggatcttgaaggaaactgaattcaaaaagatcaaagtgctgagctccggtgcgttcggcacggtgtataaggtaaggtccctggcacaggcctctgggctgggccgcagggcctctcatggtctggtggggagcccagagtccttgcaagctgtatatttccatcatctactttactctttgtttcactgagtgtttgggaaactccagtgtttttcccaagttattgagaggaaatcttttttcctagtatagcataattgagagaaaaactgatatattaaatgacataacagttatgattttgcagaaaacagatctgtatttatttcagtgttacttacctgtcttgtctttgctgatgtttcaataaaaggaattccataacttcttgctaagtcctgagcctgttttgtgtctactgttctagaaggcaaatcacagttatttcctactaggaccataggtacatcttcagagtccttaactcttttaatttgttctctgggaaagaaaaaaaagttatagcacagtcattagtaacacaaatatctttcaaaacctgtccacaacttttgtcataaaatttggctgaaagaaaacaatgtaattcctagtttccactacaccaaattttccttccaaaatgcataacaacaaagaatatgaatatggatcacatctctaccagagttaatcaactgatgcaaactcttgcacaaatgctgaaagctgtaccatacctgtctggtcttggctgaggtttcaatgaatggaatcccgtaactcttggccagttcgtgggcttgttttgtatcaactgtccttgttggcaaatcacagttgtttcccactagcaccataggtacatcatccgagtcttttactcgcttaatctgctccctaaaaacgggaatatattatcagaacataagaaaaacaagattaggctgggtacagtggctcatgcctgcaatctcagcactttgggaggctgaggagggcagactgcttgaacccaggagtttgagattagcctgggccatttcagataacttaactttcagcataattatcttgtaataagtactcatgaaaatggtcagagaaacctttatctgtatcaaagaatggtcctgcaccagtaatatgcatattaaaacaagatttacctctattgttggatcatattcgtccacaaaatgattctgaattagctgtatcgtcaaggcactcttgcctacgccatcagctccaactaccacaagtttatattcagtcattttcagcaggccttataataaaaataatgaaaatgtgactatattagaacatgtcacacataaggttaatacactatcaaatactccaccagtaccttttaatacaaactcacctttatatgaaaaattatttcaaaataccttacaaaattcaatcatg

SEQ ID NO: 4 - DNA, 1995 bp, Artificial
  Synthetic
agatcgcattcatgcgtcttcacctggaaggggtccatgtgcccctccttctggccaccatgcgaagccacactgacgtgcctctccctccctccaggaagcctacgtgatggccagcgtggacaacccccacgtgtgccgcctgctgggcatctgcctcacctccaccgtgcagctcatcacgcagctcatgcccttcggctccctcctggactatgtccgggaacacaaagacaatattggctcccagtacctgctcaactggtgtgtgcagatcgcaaaggtaatcagggaagggagatacggggaggggagataaggagccaggatcctcacatgcggtctgcgctcctgggatagcaagagtttgccatggggatatgtgtgtgcgtgcatgcagcacacacacaaaatttattgaaaatgtatttgctttttctgtaaatcatctgtgaatccagaggggaaaaatatgacaaagaaagctatataagatattattttattttacagagtaacagactagctagagacaatgaattaagggaaaatgacaaagaacagctcaaagcaatttctacacgagatcctctctctgaaatcactaagcaggagaaagattttctatggagtcacaggtaagtgctaaaatggagattctctgtttctttttctttattacagaaaaaataactgaatttggctgatctcagcatgtttttaccatacctattggaataaataaagcagaatttacatgatttttaaactataaacattgcctttttaaaaacaatggttgtaaattgaaaaaaataagaacactgatttttgtgaatactgggaactatgaaaatactatagttgagaccttcaatgactttctagtaactcagcagcatctcagggccaaaaatttaatcagtggaaaaatagcctcaattcttaccatccacaaaatggatccagacaactgttcaaactgatgggacccactccatcgagatttctctgtagctagaccaaaatcacctatttttactgtgaggtcttcatgaagaaatatatctgaggtgtagtaagtaaaggaaaacagtagatctcattttcctatcagagcaagcattatgaagagtttaggtaagagatctaatttctataattctgtaatataatattctttaaaacatagtacttcatctttcctcttaaccaaagccaaaagcagtaccatggacactggattaagaagcaatgccctctcaagagacaaaaacatttactaaatattgttttatttcctagtatagcataattgagagaaaaactgatatattaaatgacataacagttatgattttgcagaaaacagatctgtatttatttcagtgttacttacctgtcttgtctttgttgatgtttcaataaaaggaattccataacttcttgctaagtcctgagcctgttttgtgtctactgttctagaaggcaaatcacatttatttcctactaggaccataggtacatcttcagagtccttaactcttttaatttgttctctgggaaagaaaaaaaagttatagcacagtcattagtaacacaaatatctttatcagccttaggtgcggctccacagccccagtgtccctcaccttcggggtgcatcgctggtaacatccacccagatcactgggcagcatgtggcaccatctcacaattgccagttaacgtcttccttctctctctgtcatagggactctggatcccagaaggtgagaaagttaaaattcccgtcgctatcaagacatctccgaaagccaacaaggaaatcctcgatgtgagtttctgctttgctgtgtgggggtccatggctctgaacctcaggcccaccttttctcatgtctggcagctgctctgctctagaccctgctcatctccacatcctaaatgttcactttctatgtctttccctttctagctctagtgggtataact

SEQ ID NO: 5 - DNA, 20 bp, Artificial
  Synthetic
tgcctccttctgcatggtat

SEQ ID NO: 6 - DNA, 20 bp, Artificial
  Synthetic
agccaggaacgtactggtga

SEQ ID NO: 7 - DNA, 18 bp, Artificial
  Synthetic
caccgtgcagctcatcat

SEQ ID NO: 8 - DNA, 20 bp, Artificial
  Synthetic
gtctttgtgttcccggacat

SEQ ID NO: 9 - DNA, 18 bp, Artificial
  Synthetic
ctttctcttccgcaccca

SEQ ID NO: 10 - DNA, 20 bp, Artificial
  Synthetic
agccaggaacgtactggtga

SEQ ID NO: 11 - DNA, 20 bp, Artificial
  Synthetic
ccttatacaccgtgccgaac

SEQ ID NO: 12 - DNA, 20 bp, Artificial
  Synthetic
tggagcctcttacacccagt

SEQ ID NO: 13 - DNA, 16 bp, Artificial
  Synthetic
gggggttgtccacgat

SEQ ID NO: 14 - DNA, 18 bp, Artificial
  Synthetic
accatgcgaagccacact

SEQ ID NO: 15 - DNA, 15 bp, Artificial
  Synthetic
cacgctggccacgct

SEQ ID NO: 16 - DNA, 18 bp, Artificial
  Synthetic
accatgcgaagccacact

SEQ ID NO: 17 - DNA, 16 bp, Artificial
  Synthetic
gctcatgcccttcggc

SEQ ID NO: 18 - DNA, 19 bp, Artificial
  Synthetic
gcaggtactgggagccaat

SEQ ID NO: 19 - DNA, 30 bp, Artificial
  Synthetic
aagatttacctctattgttggatcatattc

SEQ ID NO: 20 - DNA, 28 bp, Artificial
  Synthetic
tgctgaaaatgactgaatataaacttgt

SEQ ID NO: 21 - DNA, 22 bp, Artificial
  Synthetic
ccctcattgcactgtactcctc

SEQ ID NO: 22 - DNA, 22 bp, Artificial
  Synthetic
ccagactgtgtttctcccttct

SEQ ID NO: 23 - DNA, 21 bp, Artificial
  Synthetic
ctcacctctatggtgggatca

SEQ ID NO: 24 - DNA, 20 bp, Artificial
  Synthetic
caggttcttgctggtgtgaa

SEQ ID NO: 25 - DNA, 24 bp, Artificial
  Synthetic
gtctctcatggcactgtactcttc

SEQ ID NO: 26 - DNA, 20 bp, Artificial
  Synthetic
cacccccaggattcttacag

SEQ ID NO: 27 - DNA, 22 bp, Artificial
  Synthetic
tcagagaagccattatctgcaa

SEQ ID NO: 28 - DNA, 26 bp, Artificial
  Synthetic
actccatagaaaatctttctcctgct

SEQ ID NO: 29 - DNA, 23 bp, Artificial
  Synthetic
gcaagaggctttggagtatttca

SEQ ID NO: 30 - DNA, 25 bp, Artificial
  Synthetic
gaagatccaatccatttttgttgtc

SEQ ID NO: 31 - DNA, 23 bp, Artificial
  Synthetic
ggtgattttggtctagctacgga

SEQ ID NO: 32 - DNA, 22 bp, Artificial
  Synthetic
tgattttggtctagctacggaa

SEQ ID NO: 33 - DNA, 23 bp, Artificial
  Synthetic
agtgattttggtctagctacgaa

SEQ ID NO: 34 - DNA, 23 bp, Artificial
  Synthetic
agtgattttggtctagctacgag

SEQ ID NO: 35 - DNA, 24 bp, Artificial
  Synthetic
catccacaaaatggatccagacaa

SEQ ID NO: 36 - DNA, 24 bp, Artificial
  Synthetic
tctgccttacagatcatgtttgag

SEQ ID NO: 37 - DNA, 20 bp, Artificial
  Synthetic
ccagaggcgtacagggatag

SEQ ID NO: 38 - DNA, 20 bp, Artificial
  Synthetic
tttggccggcccaaaatctg

SEQ ID NO: 39 - DNA, 25 bp, Artificial
  Synthetic
gcgacgggaattttaactttctcac

SEQ ID NO: 40 - DNA, 20 bp, Artificial
  Synthetic
gctcagcacgcagctcatgc

SEQ ID NO: 41 - DNA, 21 bp, Artificial
  Synthetic
tgtgatcttgacatgctgcgg

SEQ ID NO: 42 - DNA, 26 bp, Artificial
  Synthetic
cagtttccttcaagatcctcaagaga

SEQ ID NO: 43 - DNA, 19 bp, Artificial
  Synthetic
tggagggagggagaggcac

SEQ ID NO: 44 - DNA, 19 bp, Artificial
  Synthetic
tggagggagggagaggcac

SEQ ID NO: 45 - DNA, 25 bp, Artificial
  Synthetic
ctatgtccgggaacacaaagacaat

SEQ ID NO: 46 - DNA, 25 bp, Artificial
  Synthetic
tgaattagctgtatcgtcaaggcac

SEQ ID NO: 47 - DNA, 28 bp, Artificial
  Synthetic
ccaagagacaggtttctccatcaattac

SEQ ID NO: 48 - DNA, 26 bp, Artificial
  Synthetic
agtggttctggattagctggattgtc

SEQ ID NO: 49 - DNA, 29 bp, Artificial
  Synthetic
caaacaggtttcaccatctataaccactt

SEQ ID NO: 50 - DNA, 26 bp, Artificial
  Synthetic
acgagatcctctctctaaaatcactg

SEQ ID NO: 51 - DNA, 25 bp, Artificial
  Synthetic
tgaatgatgcacgtcatggtggctg

SEQ ID NO: 52 - DNA, 25 bp, Artificial
  Synthetic
caaactgatgggacccactccatcg

SEQ ID NO: 53 - DNA, 25 bp, Artificial
  Synthetic
ccatgtacgttgctatccaggctgt

SEQ ID NO: 54 - DNA, 18 bp, Artificial
  Synthetic
  modified_base (1)..(18): D-Lys modified
ggccagcccaaaatctgt

SEQ ID NO: 55 - DNA, 15 bp, Artificial
  Synthetic
  modified_base (1)..(15): D-Lys modified
acccagcagtttggc

SEQ ID NO: 56 - DNA, 25 bp, Artificial
  Synthetic
  modified_base (1)..(25): D-Lys modified
cggagatgttgcttctcttaattcc

SEQ ID NO: 57 - DNA, 14 bp, Artificial
  Synthetic
  modified_base (1)..(14): Acetyl modified
tcatcacgcagctc

SEQ ID NO: 58 - DNA, 19 bp, Artificial
  Synthetic
  modified_base (1)..(19): D-Lys modified
cggagcccagcactttgat

SEQ ID NO: 59 - DNA, 17 bp, Artificial
  Synthetic
  modified_base (1)..(17): C3 spacer chemical modification
ttgtccacgctggccat

SEQ ID NO: 60 - DNA, 17 bp, Artificial
  Synthetic
  modified_base (1)..(17): C3 spacer chemical modification
ttgtccacgctggccat

SEQ ID NO: 61 - DNA, 16 bp, Artificial
  Synthetic
ttcggctgcctcctgg

SEQ ID NO: 62 - DNA, 24 bp, Artificial
  Synthetic
  modified_base (1)..(24): D-Lys modified
ctacgccaccagctccaactacca

SEQ ID NO: 63 - DNA, 23 bp, Artificial
  Synthetic
  modified_base (1)..(23): D-Lys modified
tgtactcctcttgacctgctgtg

SEQ ID NO: 64 - DNA, 25 bp, Artificial
  Synthetic
  modified_base (1)..(25): D-Lys modified
caacaccacctgctccaaccaccac

SEQ ID NO: 65 - DNA, 23 bp, Artificial
  Synthetic
  modified_base (1)..(23): D-Lys modified
ggcactgtactcttcttgtccag

SEQ ID NO: 66 - DNA, 21 bp, Artificial
  Synthetic
  modified_base (1)..(21): D-Lys modified
agatcctctctctgaaatcac

SEQ ID NO: 67 - DNA, 23 bp, Artificial
  Synthetic
  modified_base (1)..(23): D-Lys modified
aatgatgcacatcatggtggctg

SEQ ID NO: 68 - DNA, 24 bp, Artificial
  Synthetic
  modified_base (1)..(24): D-Lys modified
atcgagatttcactgtagctagac

SEQ ID NO: 69 - DNA, 16 bp, artificial
  Synthetic
ttcggctgcctcctgg

SEQ ID NO: 70 - DNA, 16 bp, artificial
  Synthetic
  modified_base (1)..(16): D-Lysine, oxy-aza and aza-aza modified
ttcggctgcctcctgg

SEQ ID NO: 71 - DNA, 19 bp, artificial
  Synthetic
  modified_base (1)..(19): D-Lysine, oxy-aza and aza-aza modified
cggagcccagcactttgat

SEQ ID NO: 72 - DNA, 25 bp, artificial
  Synthetic
  modified_base (1)..(25): D-Lysine, oxy-aza and aza-aza modified
cggagatgttgcttctcttaattcc

SEQ ID NO: 73 - DNA, 14 bp, artificial
  Synthetic
  modified_base (1)..(14): D-Lysine, oxy-aza and aza-aza modified
tcatcacgcagctc

SEQ ID NO: 74 - DNA, 18 bp, artificial
  Synthetic
  modified_base (1)..(18): D-Lysine, oxy-aza and aza-aza modified
ggccagcccaaaatctgt

SEQ ID NO: 75 - DNA, 25 bp, artificial
  Synthetic
  modified_base (1)..(25): D-Lysine, oxy-aza and aza-aza modified
caacaccacctgctccaaccaccac

SEQ ID NO: 76 - DNA, 25 bp, artificial
  Synthetic
  modified_base (1)..(25): D-Lysine, oxy-aza and aza-aza modified
ttcttgtccagctgtatccagtatg

SEQ ID NO: 77 - DNA, 24 bp, artificial
  Synthetic
  modified_base (1)..(24): D-Lysine, oxy-aza and aza-aza modified
ctacgccaccagctccaactacca

SEQ ID NO: 78 - DNA, 21 bp, artificial
  Synthetic
  modified_base (1)..(21): D-Lysine, oxy-aza and aza-aza modified
ctcttgacctgctgtgtcgag

SEQ ID NO: 79 - DNA, 15 bp, artificial
  Synthetic
  modified_base (1)..(15): D-Lysine, oxy-aza and aza-aza modified
tgtctttgctgatgt

SEQ ID NO: 80 - DNA, 24 bp, artificial
  Synthetic
  modified_base (1)..(24): D-Lysine, oxy-aza and aza-aza modified
ctgacctagttccaatcttttctt

SEQ ID NO: 81 - DNA, 23 bp, artificial
  Synthetic
  modified_base (1)..(23): D-Lysine, oxy-aza and aza-aza modified
aatgatgcacatcatggtggctg

SEQ ID NO: 82 - DNA, 15 bp, artificial
  Synthetic
  modified_base (1)..(15): D-Lysine, oxy-aza and aza-aza modified
ctccttctctgagtg

SEQ ID NO: 83 - DNA, 24 bp, artificial
  Synthetic
  modified_base (1)..(24): D-Lysine, oxy-aza and aza-aza modified
atcgagatttcactgtagctagac

(19) United States
(12) Patent Application Publication
(10) Pub. No.: US 2022/0025453 A1
(43) Pub. Date: Jan. 27, 2022

(54) XENONUCLEIC ACID-MEDIATED MULTIPLEX QPCR CLAMPING TECHNOLOGY FOR LUNG CANCER MUTATION DETECTION

(71) Applicants: Michael Y Sha, Castro Valley, CA (US); Maidar Jamba, Concord, CA (US); Qing Sun, Fairfield, CA (US); Michael J Powell, Alamo, CA (US); Aiguo Zhang, San Ramon, CA (US)

(72) Inventors: Michael Y Sha, Castro Valley, CA (US); Maidar Jamba, Concord, CA (US); Qing Sun, Fairfield, CA (US); Michael J Powell, Alamo, CA (US); Aiguo Zhang, San Ramon, CA (US)

(21) Appl. No.: 17/384,635

(22) Filed: Jul. 23, 2021

Related U.S. Application Data
(60) Provisional application No. 63/055,884, filed on Jul. 23, 2020.

(51) Int. Cl. C12Q 1/6858; C12Q 1/6827; C12Q 1/686

(52) U.S. Cl. CPC C12Q 1/6858; C12Q 1/6827; C12Q 2600/16; C12Q 2600/156; C12Q 1/686

(57) ABSTRACT

The invention provides a multiplex method for enriching a plurality of target polynucleotide sequences containing genetic mutations associated with lung cancer comprising: (a) providing a biological sample; (b) isolating DNA from said sample; said DNA including said plurality of target polynucleotide sequences containing genetic mutations; (c) providing a plurality of primer probes targeted to said target polynucleotide sequences said primer probes allowing formation of a PCR product; (d) providing a plurality of target specific xenonucleic acid clamps oligomer probes specific for wildtype polynucleotide sequences; so that during the qPCR process only mutant templates are amplified: (e) admixing the plurality of primer probes and the plurality of xenonucleic clamping probes with the target nucleic acid sample; (f) performing a PCR amplification process in reaction solution under hybridization conditions thereby generating multiple amplicons; and (g) detecting said amplicons and wherein said xenonucleic acid clamps have aza-aza, thio-aza and oxy-aza chemical functionality.

This application claims the priority benefit under 35 U.S.C. section 119 of U.S. Provisional Patent Application No. 63/055,884 entitled “A Xenonucleic Acid-Mediated Multiplex qPCR Clamping Technology For Lung Cancer Mutation Detection” filed Jul. 23, 2020, which is in its entirety herein incorporated by reference.

### FIELD OF THE INVENTION

The present invention relates to DNA mutation detection. The invention further relates to enrichment of mutant polynucleotide sequences. The present invention further relates to minimally invasive sampling and analysis of mutations in clinical samples.

The disclosed inventions relate generally to methods for detecting nucleic acid mutations and fusions using amplification methods such as the polymerase chain reaction (PCR). The present invention also relates to the field of medicine and more specifically to neoplastic lung diseases. The invention also relates to the field of molecular biology, more particular to the detection of cancer associated mutations in a biological sample. The invention also relates to the field of biomarker analysis, particularly determining genomic alterations from biological samples, including plasma samples.

The instant invention also relates to a method for determining whether a target polynucleotide sequence contained in a nucleic acid sample has nucleotide variation(s) in a selected region thereof, the steps of which involve the use of a pair of primers that allows the formation of a PCR product having a sequence covering that of the selected region of the target polynucleotide sequence via a PCR process, and a xeno nucleic acid (XNA) that acts as a PCR clamp as well as a sensor probe. This invention also relates to a kit for use in determining the presence of nucleotide variation(s) in the target polynucleotide sequence, which comprises the pair of primers and the XNA.

The present embodiments relate to precision molecular diagnostics, and in particular, to compositions in detecting sequence variants, such as SNPs, insertions deletions, and altered methylation patterns, from samples. The embodiments disclosed herein can be used to detect (and quantify) sequence variants present in samples that include an excess of wild-type sequences.

The present technology further relates to methods for determining whether a patient diagnosed with lung cancer will benefit from or is predicted to be responsive to treatment with a therapeutic agent alone or in a specific combination with other therapeutic agents. These methods are based on screening a bilogical sample from a patient and detecting alterations in target nucleic acid sequences corresponding to a specific set of cancer-related genes. Kits for use in practicing the methods are also provided.

In some embodiments, the disclosure relates generally to methods, compositions, systems, apparatuses and kits for amplifying one or more target sequences within a sample containing a plurality of target sequences especially lung cancer associated sequences containign mutations. Optionally, a plurality of target sequences, for example at least 10, 50, 100, 500, 1000, 2500, 5000, 7500, 10000, 25000, 50000 or 100000, are amplified within a single amplification reaction. In some embodiments, the disclosure relates generally to methods, compositions, systems, apparatuses and kits for amplifying one or more target sequences from a single source, such as a biological sample containing DNA mutations or formalin-fixed paraffin-embedded (FFPE) DNA. In particular, methods, kits, systems apparatuses and compositions useful for amplifying one or more target sequences using XNA's to clamp and prevent wiltype genes from amplifying while amplifying the DNA containing mutations.

### BACKGROUND OF THE INVENTION

Lung cancer is the leading cause of cancer-related mortality throughout the world. Approximately 80%-85% of all lung cancers are non-small cell lung cancer (NSCLC), which include squamous cell carcinoma, adenocarcinoma, and large-cell carcinoma. Current treatment options include surgical resection, platinum-based doublet chemotherapy with 3^{rd} generation chemotherapy including gemcitabine, docetaxel, paclitaxel, vinorelbine, irinotecan, or pemetrexed, and radiation therapy alone or in combination. Despite these therapies, the disease is rarely curable and prognosis is dismal, with an overall 5-year survival rate of only 15%.

Most NSCLCs are diagnosed at an advanced stage, are clinically aggressive, and have a high metastatic potential. Additionally, current NSCLC chemotherapeutic regimens have low efficacy. For example, patients with untreated advanced NSCLC have a median survival of 7-15 months, while those treated with current platinum-based doubled chemotherapy regimens have an 8-12 month median survival. Research into the mechanisms of carcinogenesis and malignant progression of NSCLC has revealed that different driver mutations are altered in this human malignancy, and targeted therapies based on certain genetic alterations in NSCLC tumors have been developed. Identifying mutations in oncogenes associated with non-squamous NSCLC can help determine which patients are more likely to benefit from a targeted therapy. Such oncogenes include EGFR, KRAS, BRAF, PIK3CA, ROS1 and ALK, and molecular diagnostic testing for ALK, ROS1 and EGFR mutations is now recommended for NSCLCs to guide therapy.

Fusion between echinoderm microtubule-associated protein like 4 (EML4) and ALK is seen in approximately 2-7% of patients with NSCLC adenocarcinomas. This and other ALK gene fusions are more common in nonsmokers or light smokers and in those with adenocarcinomas. Because EGFR, ROS1 and ALK mutations are mutually exclusive, patients with ALK rearrangements (gene fusion) are not thought to benefit from treatments targeting the other mutations. For example, EGFR-targeting tyrosine kinase inhibitors (TKIs). Instead, treatment with an ALK inhibitors such as crizotinib (Xalkori), ceritinib (Zykadia), or brigatinib (Alunbrig), are indicated. In patients who received crizotinib as second-line therapy, the 1-year overall survival rate was 70% and the 2-year overall survival rate was 55%. By contrast, ALK-positive matched controls had a 1-year survival of 44% and a 2-year survival of 12%, whereas ALK-negative controls had a 1-year survival of 47% and a 2-year survival of 32%. These data suggest that the presence of the ALK gene fusion itself does not confer a poorer outcome but that the use of crizotinib in ALK-positive patients can improve outcomes. Kwak et al, Anaplastic lymphoma kinase inhibition in non-small-cell lung cancer. N. Engl. J. Med., 363(18):1693-703 (2010); Crin et al, Initial phase II results with crizotinib in advanced ALK-positive non-small cell lung cancer (NSCLC): PROFILE 1005, J. Clin. Oncol., 29(suppl 15):Abstract 7514 (2011); Shaw et al, Ceritinib in ALK-rearranged non-small-cell lung cancer, N. Engl. J. Med., 370(13):1189-97 (2014). Based on such data, testing for ALK rearrangement is recommended in patients with metastatic NSCLC adenocarcinoma, and the ALK inhibitor crizotinib is recommended for ALK-gene fusion positive patients.

Similarly, proto-oncogene tyrosine-protein kinase ROS (ROS1) is an orphan receptor tyrosine kinase (RTK) that forms fusions and defines another clinically actionable oncogenic driver mutation in NSCLC. It has been recently reported that approximately 1.4% of NSCLCs harbor ROS1 rearrangements. Of the ROS1 fusion-positive tumors, 30% are known to harbor a recurrent translocation t[5;6][q32;q22], which creates the CD74 molecule, major histocompatibility complex, class II invariant chain (CD74)-ROS fusion kinase. ROS1 is evolutionarily related to ALK, and ALK inhibitors can also be used on ROS1-fusion positive cancers according to their similarity.

Somatic gain-of-function RET mutations have been observed in 30-50% of sporadic medullary thyroid cancer, and somatic RET gene fusions have been observed in 30-50% of sporadic papillary thyroid cancer. The US Food and Drug Administration (FDA) has approved two inhibitory drugs, vandetanib (ZD6474) and cabozantinib (XL184), for the treatment of advanced medullary thyroid cancer. The RET fusions are present in 1-2% of NSCLC adenocarcinoma of patients of both Asian and European descent. Several studies indicate that RET fusion occurs preferentially in young non-smoker and light-smoker patients.

These examples demonstrate the value in the ability to determine if an individual harbors one or more gene fusions which influence the effectiveness of specific therapeutic treatments, particularly in treatment of cancers such as NSCLC. Fluorescent in situ hybridization (FISH) is currently the reference detection method for ALK fusions. This technique uses two specific DNA probes, each coupled to a fluorescent marker, one green and one red, which cover the 2p23 ALK region. In the wild-type scenario, the red signal (3′ ALK) and the green signal (5′ ALK) are adjacent. When the distance between these two signals is more than twice the signal diameter, they are considered separated, reflecting a physical separation of the two DNA regions and hence a translocation (gene fusion).

FISH is considered to be positive for a translocation if >15% of the tumor cells counted in four fields show either a separation between the green and red signals or a single red signal with loss of the associated green signal. This 15% threshold allows for errors due to background noise, reading or aberrant hybridization. At least 50 cells must be counted, with a second count of another 50 cells by a second reader if there are between 10% and 50% positive cells. The strengths of FISH lie in its ability to detect ALK rearrangements irrespective of the variant or the fusion protein, as well as its correlation with clinical efficacy. It has been approved by the FDA for use in connection with crizotinib therapy (Vysis ALK Break-Apart FISH Probe kit; Abbott Molecular, Inc., Des Plaines, Ill., USA). However, the use of FISH analysis for detecting ALK translocations can be challenging as 1) the technique is relatively expensive, 2) accurate interpretation of the results requires the expertise and experience of a trained cytologist who must view testing of multiple tissue sections, 3) the technique does not identify specific translocation types, and 4) the technique often has a lengthy turn-around time.

Immunohistochemistry (IHC) is another method for detecting ALK rearrangements in lung cancer. Initially, IHC encountered sensitivity issues and occasional false-positive results. However, newer ultrasensitive IHC techniques appear to offer a more reliable and sensitive screening method. The positivity threshold is typically visual, requiring moderate to intense staining in 5-10% of cells. Advantages of IHC are mainly its low cost in terms of both time and manpower, but standardization of the test is difficult. The challenges in developing IHC for ALK detection in NSCLC are: 1) tissue preparation, 2) antibody choice, 3) signal enhancement systems, and 4) the optimal scoring system. While IHC is a reliable screening tool, FISH confirmation is required in the event of positive IHC and even in some cases for negative IHC in patients presenting predictive rearrangement markers, including younger age, light smokers (10 pack-years) and testing negative for other mutations, notably EGFR and KRAS.

Polymerase chain reaction (PCR) is a widely used technique for the detection of pathogens. The technique uses a DNA polymerase used to amplify a piece of DNA by in vitro enzymatic replication. The PCR process generates DNA that is used as a template for replication. This results in a chain reaction that exponentially amplifies the DNA template.

Reverse transcriptase polymerase chain reaction (RT-PCR) and quantitative RT-PCR (qRT-PCR) are also used as diagnostic techniques for ALK translocations. Typically, RNA is converted into cDNA by reverse transcriptase and the cDNA is PCR amplified with specific primers. See, for example, Sanders et al, U.S. Pat. No. 9,175,350 B2, and Begovich et al, US 2016/0304937 A1. Amplification requires primer sets specific for each translocation. This highly specific technique offers the additional advantage of identifying the fusion gene associated with ALK. Its limited use to date is due to the requirement of a quality DNA sample from a formalin-fixed, paraffin-embedded (FFPE) tissue sample or from fresh or frozen tumor tissue. Additionally, detection of fusion genes by RT-PCR or qRT-PCR might not be successful because of the highly variable nature of gene fusions.

Technologies for genomic detection most commonly use DNA probes to hybridize to target sequences. To achieve required sensitivity, the use of PCR to amplify target sequences has remained standard practice in many labs. While PCR has been the principle method to identify genes associated with disease states, the method has remained confined to use within a laboratory environment. Most current diagnostic applications that can be used outside of the laboratory are based on antibody recognition of protein targets and use ELISA-based technologies to signal the presence of a disease. These methods are fast and fairly robust, but they can lack the specificity associated with nucleic acid detection.

With the advent of molecular diagnostics and the discovery of numerous nucleic acid biomarkers useful in the diagnosis and treatment of conditions and diseases, detection of nucleic acid sequences, and sequence variants, mutations and polymorphisms has become increasingly important. In many instances, it is desirable to detect sequence variants or mutations (which may in some instances, differ by one a single nucleotide) present in low copy numbers against a high background of wild-type sequences. For example, as more and more somatic mutations are shown to be biomarkers for cancer prognosis and prediction of therapeutic efficacy, the need for efficient and effective methods to detect rare mutations in a sample is becoming more and more critical. In the case in which one or more allelic variants is/are present in low copy number compared to wild-type sequences, the presence of excess wild-type target sequence creates challenges to the detection of the less abundant variant target sequence. Nucleic acid amplification/detection reactions almost always are performed using limiting amounts of reagents. A large excess of wild-type target sequences, thus competes for and consumes limiting reagents. As a result amplification and/or detection of rare mutant or variant alleles under these conditions is substantially suppressed, and the methods may not be sensitive enough to detect the rare variants or mutants. Various methods to overcome this problem have been attempted. These methods are not ideal, however, because they either require the use of a unique primer for each allele, or the performance of an intricate melt-curve analysis. Both of these shortcomings limit the ability and feasibility of multiplex detection of multiple variant alleles from a single sample.

Increasing knowledge of the genetic and epigenetic changes occurring in cancer cells provides an opportunity to detect, characterize, and monitor tumors by analyzing tumor-related nucleic acid sequences and profiles. These changes can be observed by detecting any of a variety of cancer-related biomarkers. Various molecular diagnostic assays are used to detect these biomarkers and produce valuable information for patients, doctors, clinicians and researchers. So far, these assays primarily have been performed on cancer cells derived from surgically removed tumor tissue or from tissue obtained by biopsy.

Detection of mutations associated with lung cancer whether prior to diagnosis, in making a diagnosis, for disease staging or to monitor treatment efficacy has traditionally relied or solid tumor biopsy samples. Such sampling is highly invasive and not without risk of potentially contributing to metastasis or surgical complications. Better and less invasive methods are needed for detecting mutations associated with cancer.

Accordingly, a need exists for a simple, high-throughput method for detecting the genetic mutations/variations that are in a gene selected from the group consisting of: EGFR gene (codons 858, 746, 790, 861, 719, 768, and 769), KRAS gene (codons 12 and 61), NRAS gene (codons 12 and 61), PIK3CA (codons 542 and 1047), and BRAF (codon 600) associated with the non-small cell lung cancer (NSCLC).

### BRIEF DESCRIPTION OF THE FIGURES

FIG. 1 illustrates the mechanism of the XNA clamping process.

FIG. 2 shows the differential melting temperature (Tm) between the XNA clamp bound to mutant templates vs wild type templates.

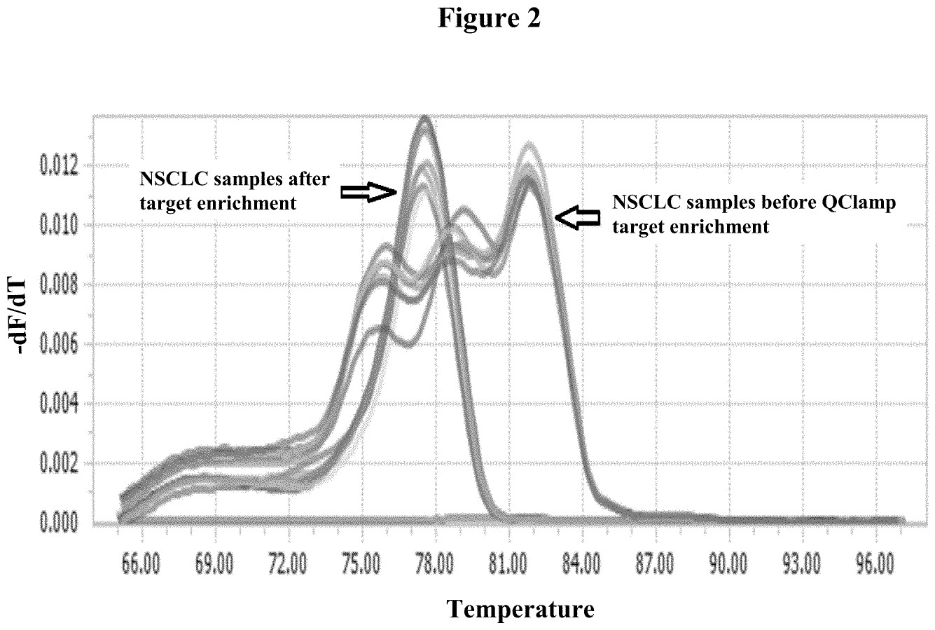

FIG. 3 show specific hydrolysis probe having a different fluorophore (and quencher) selected from the available fluorophores for multiplex applications.

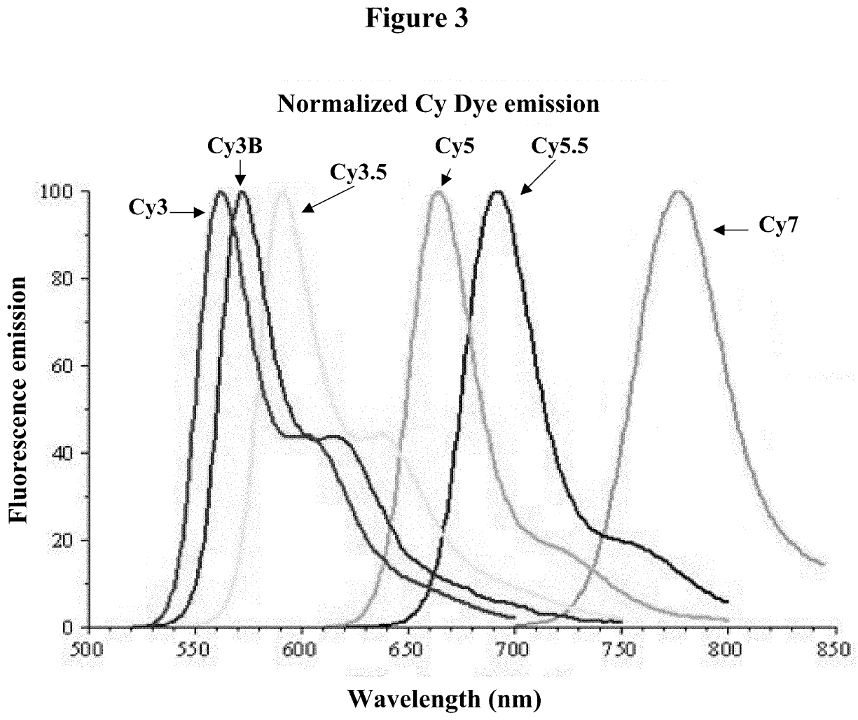

FIG. 4 is a representative fluorophore spectral data and quencher selection guide.

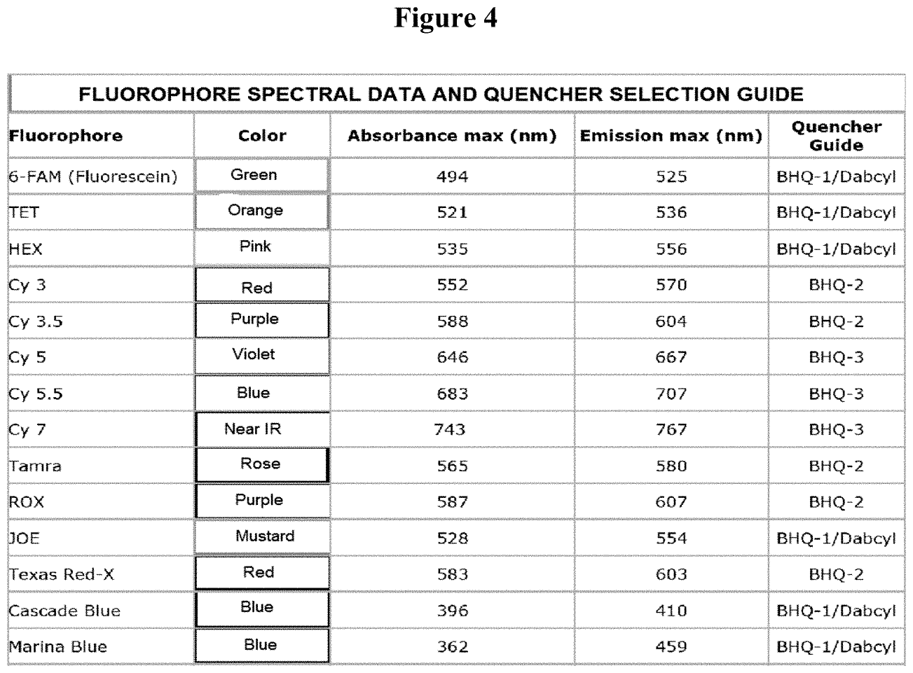

FIG. 5 shows a specific locus specific hydrolysis probe assay.

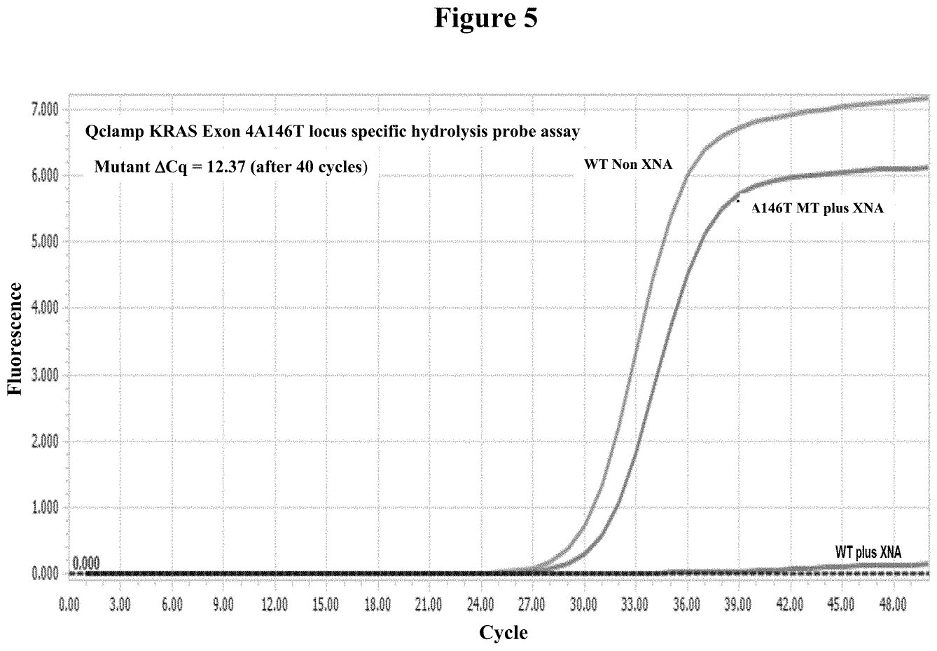

FIG. 6 is a schematic illustrating how circulating tumor cells (CTC's) and cell-free DNA (cfDNA) derived from tumor cells are present in the peripheral blood of cancer patients.

### SUMMARY OF THE INVENTION

Detection of rare sequence mutations/variants in biological samples presents numerous challenges. The methods and kits disclosed herein provide for improved, efficient means to detect rare mutations in lung cancer patients within a high background of wild-type allelic sequences using real-time amplification methods.

The invention provides multiplex method for enriching a plurality of target polynucleotide sequences containing genetic mutations/variations associated with lung cancer, said method comprising: (a) providing a biological sample; (b) isolating DNA from said biological sample; said DNA including said plurality of target polynucleotide sequences containing genetic variations associated with lung cancer; (c) providing a plurality of primer probes targeted to said plurality of target polynucleotide sequences said primer probes allowing formation of a PCR process product; (d) providing a plurality of target specific xenonucleic acid clamps oligomer probes specific for wildtype polynucleotide sequences; so that during the qPCR process only mutant templates are amplified and wherein said xenonucleic acid clamps have aza-aza and oxy-aza chemical functionality; (e) admixing the plurality of primer probes and the plurality of xenonucleic clamping probes with the target nucleic acid sample; (f) performing a PCR amplification process in a reaction solution under hybridization conditions thereby generating multiple amplicons; and (g) detecting said amplicons.

Of particular interest are target polynucleotide sequences containing genetic mutations/variations in a gene selected from the group consisting of: EGFR gene (codons 858, 746, 790, 861, 719, 768, and 769), KRAS gene (codons 12 and 61), NRAS gene (codons 12 and 61), PIK3CA (codons 542 and 1047), and BRAF (codon 600) associated with the non-small cell lung cancer (NSCLC).

The invention further provides a method for enriching a plurality of multiple target polynucleotide sequences containing a genetic mutations/variations said method comprising: (a) providing a biological sample; (b) isolating DNA from said biological sample; said DNA including said plurality of multiple target polynucleotide sequences containing genetic mutations/variations; (c) providing a library of amplifying primer probes targeted to said plurality of multiple target polynucleotide sequences containing a genetic mutations/variations; said primer probes allowing formation of PCR process products; (d) providing a library of target specific xenonucleic acid clamp oligomer probes specific for a plurality of multiple wildtype polynucleotide sequences so that during the qPCR process only mutant templates are amplified and wherein said xenonucleic acid clamps have aza-aza and oxy-aza chemical functionality; (e) admixing the primer probes and the xenonucleic clamping probes with the multiple target nucleic acid samples; (f) performing a PCR amplification process in a reaction solution under hybridization conditions thereby generating multiple amplicons; and (g) detecting said amplicons.

The invention is additionally directed to a multiplex method for conducting minimally invasive biopsies in a mammalian subjects suspected of a having lung cancer, said method comprising: (a) providing biological samples derived from said mammalian subjects; (b) isolating DNA from said biological samples; said DNA including a plurality of multiple target polynucleotide sequences containing genetic mutations/variations; (c) providing a library of amplifying primer probes targeted to said plurality of multiple target poly-nucleotide sequences containing genetic mutations/variations; said primer probes allowing formation of PCR process products; (d) providing a library of target specific xenonucleic acid clamp oligomer probes specific for a plurality of multiple wildtype polynucleotide sequences so that during the qPCR process only mutant templates are amplified and wherein said xenonucleic acid clamps have aza-aza and oxy-aza chemical functionality; (e) admixing the primer probes and the xenonucleic clamping probes with the plurality of multiple target nucleic acid samples; (f) performing a PCR amplification process in a reaction solution under hybridization conditions thereby generating multiple amplicons; and (f) detecting said amplicons.

The invention is also directed to means and methodology for the rapid isolation of genetic material from biological fluids and the sensitive detection of somatic and germ-line mutations present in circulating cells and cell-free genetic material obtained from biological fluids using gene amplification and xeno-nucleic acid (XNA) clamping.

This invention also provides a method for determining whether a target polynucleotide sequence contained in a nucleic acid sample has nucleotide mutations/variations in a selected region thereof, comprising the steps of: providing a pair of a first primer and a second primer which allows the formation of a PCR product having a sequence covering that of the selected region of the target polynucleotide sequence via a PCR process, the first primer having a sequence identical to that of a first region located upstream of the selected region of the target polynucleotide sequence, the second primer having a sequence based on that of a second region located downstream of the selected region of the target polynucleotide sequence, wherein the 6′-end of the sequence of the first region is spaced apart from the 5′-end of the sequence of the sequence of the selected region by 30 nucleotides or more; providing a detectable xenonucleic acid probe having a sequence that complements fully the sequence of the selected region of the target polynucleotide sequence having no nucleotide variation(s) therein, such that hybridization of the detectable xenonucleic acid probe to the selected region of the target polynucleotide sequence having no nucleotide variation(s) results in the formation of a duplex having a melting temperature; determining the melting temperature of the duplex; admixing the detectable xenonucleic acid probe and the pair of the first primer and the second primer with the nucleic acid sample to form a mixture; subjecting the mixture to a PCR process including an extension reaction set to run at a temperature lower than the melting temperature of the duplex by 5 to 20° C., such that a mixture of PCR products is obtained; and subjecting the mixture of PCR products thus-obtained to a melting analysis to determine melting temperatures of the PCR products, wherein the presence of at least one melting temperature lower than the melting temperature of the duplex is indicative of the nucleotide variation(s) in the selected region of the target polynucleotide sequence contained in the nucleic acid sample.

The invention also provides a kit for determining whether a target polynucleotide sequence contained in a nucleic acid sample has nucleotide mutations/variation(s) in a selected region thereof, comprising: a detectable xenonucleic acid probe having a sequence that complements fully the sequence of the selected region of the target polynucleotide sequence having no nucleotide variation(s) therein, such that hybridization of the detectable xenonucleic acid probe to the selected region of the target polynucleotide sequence having no nucleotide variation(s) results in the formation of a duplex having a melting temperature; a pair of a first primer and a second primer which allows the formation of a PCR product having a sequence covering that of the selected region of the target polynucleotide sequence via a PCR process, the first primer having a sequence identical to that of a first region located upstream of the selected region of the target polynucleotide sequence, the second primer having a sequence based on that of a second region located downstream of the selected region of the target polynucleotide sequence, wherein the 5′-end of the sequence of the first region is spaced apart from the 5′-end of the sequence of the sequence of the selected region by 30 nucleotides or more; and an instruction sheet providing guidance for a user to use the detectable xenonucleic acid probe and the pair of the first primer and the second primer in a method as described above.

The invention provides a Lung Cancer Multiplex Taqman Assay for qualitative detection of mutations in the EGFR gene (codons 858, 746, 790, 861, 719, 768, and 769), KRAS gene (codons 12 and 61), NRAS gene (codons 12 and 61), PIK3CA (codons 542 and 1047), and BRAF (codon 600) associated with the non-small cell lung cancer (NSCLC).

### DETAILED DESCRIPTION OF THE INVENTION

It is to be understood that both the foregoing general description and the following detailed description are exemplary and explanatory only and are not intended to limit the scope of the current teachings. In this application, the use of the singular includes the plural unless specifically stated otherwise. Also, the use of “comprise”, “contain”, and “include”, or modifications of those root words, for example but not limited to, “comprises”, “contained”, and “including”, are not intended to be limiting. Use of “or” means “and/or” unless stated otherwise. The term “and/or” means that the terms before and after can be taken together or separately. For illustration purposes, but not as a limitation, “X and/or Y” can mean “X” or “Y” or “X and Y”. Whenever a range of values is provided herein, the range is meant to include the starting value and the ending value and any value or value range there between unless otherwise specifically stated. For example, “from 0.2 to 0.5” means 0.2, 0.3, 0.4, 0.5; ranges there between such as 0.2-0.3, 0.3-0.4, 0.2-0.4; increments there between such as 0.25, 0.35, 0.225, 0.335, 0.49; increment ranges there between such as 0.26-0.39; and the like.

In a first embodiment, the present invention relates to compositions and methods for the selective enrichment of low-abundance polynucleotides in a sample derived from lung cancer patients. These methods use xeno-nucleic acid (XNA) nucleobase oligomers having aza-aza and oxy-aza chemical functionality to selectively block DNA polymerase activity on high abundance wild-type DNA templates, thereby resulting in enrichment of less abundant mutated DNA templates present in the biological sample during a polymerase chain reaction (PCR). The methodology of the present invention can be used to improve DNA sequencing (Sanger sequencing and Pyrosequencing) and also enhance cDNA library preparation for next generation DNA sequencing (NGS).

Utilizing xeno-nucleic acid (XNA) clamping probes in the PCR mediated amplification of DNA templates, only target genetic material that has a mutation or variation, e.g. single nucleotide polymorphism (SNP), gene deletion or insertion and/or translocation or truncation is amplified in the oligonucleotide primer directed polymerase chain reaction (qPCR).

The XNA probe clamping sequences are designed to bind specifically by Watson-Crick base pairing to abundant wild-type sequences in the DNA templates derived from the biological sample of interest. The presence of the XNA probes in the PCR primer mix employed for the target amplification reaction causes inhibition of the polymerase mediated amplification of wild-type templates but does not impede the amplification of mutant template sequences.

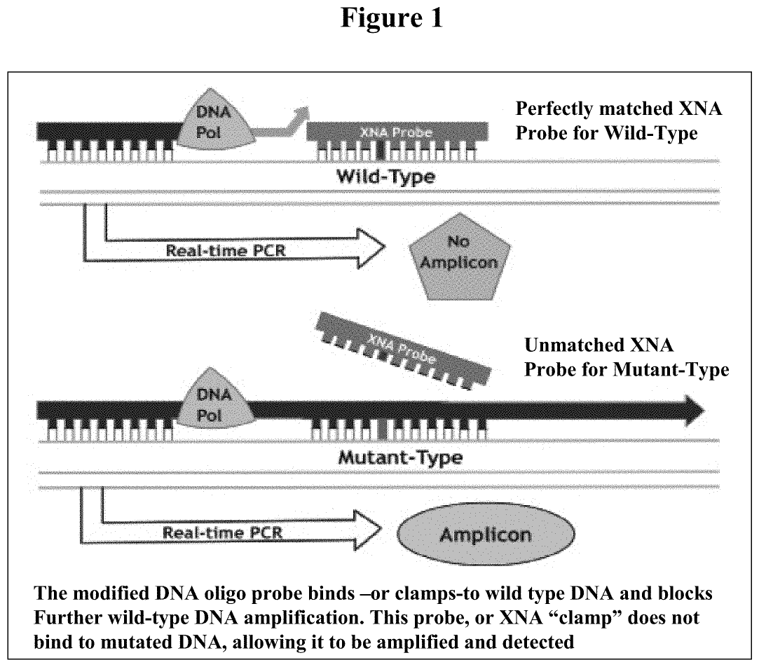

The mechanism of the XNA clamping process is depicted in FIG. 1. As shown in FIG. 1, the modified DNA oligo probe binds or clamps to wild type DNA and blocks further wild type amplification. This probe or XNA “clamp” does not bind to mutated DNA, allowing it to be amplified and detected.

The suppression of wild-type (wt) template amplification and amplification of only mutant templates is achieved because there is a differential melting temperature (Tm) between the XNA clamp bound to mutant templates vs wild type templates:

Tm(XNA mutant template)<<Tm(XNA wt template)

The Tm differential is as much as 15-20° C. for the XNA clamp probes. So that during the PCR process only mutant templates are amplified.

For purposes of illustration, the scheme below illustrates the differences between DNA and XNA:

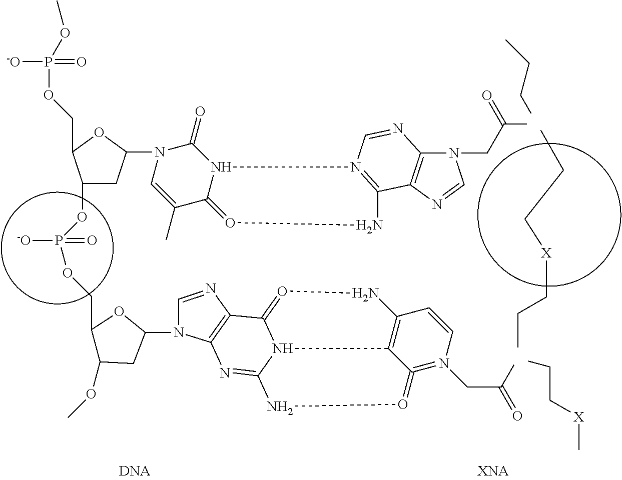

Applicant has developed a multitude of XNA chemistry and multiple applications of XNA in molecular testing including, PCR-Clamping, in-situ detection of gene mutations and targeted CRISPR/Cas9 gene-editing and detection. Applicant's XNA chemistry is unique in that a single nucleotide change in the target sequence can lead to a melting temperature differential of as much as 15-200C. For natural DNA the Tm differential for such a change is only 5-70C.

Representative examples are shown below:

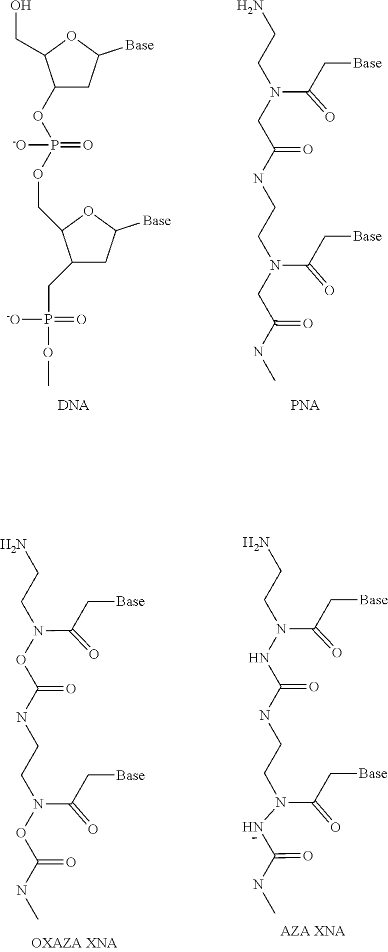

The XNA monomers are synthesized as shown in the following schemes:

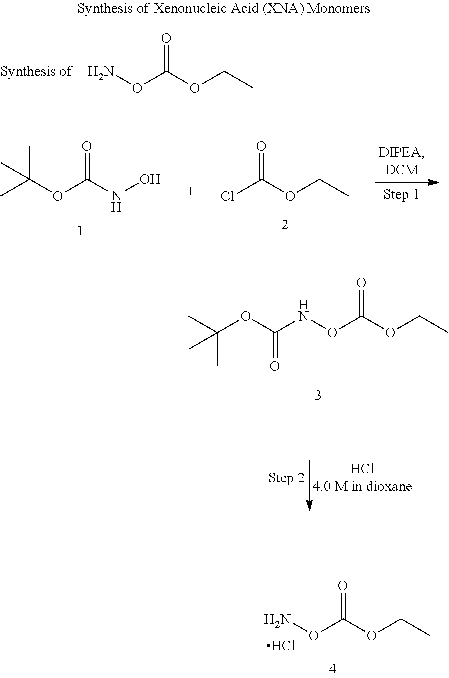

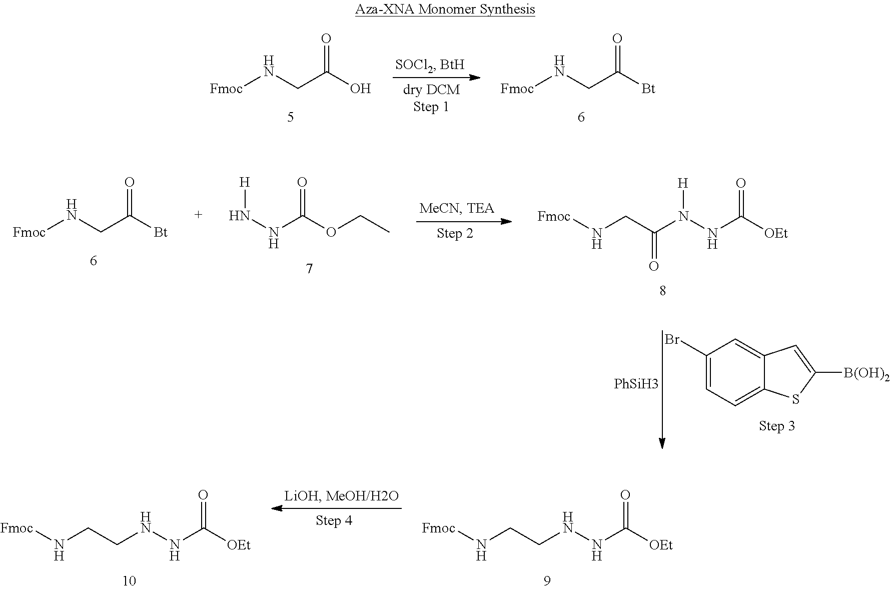

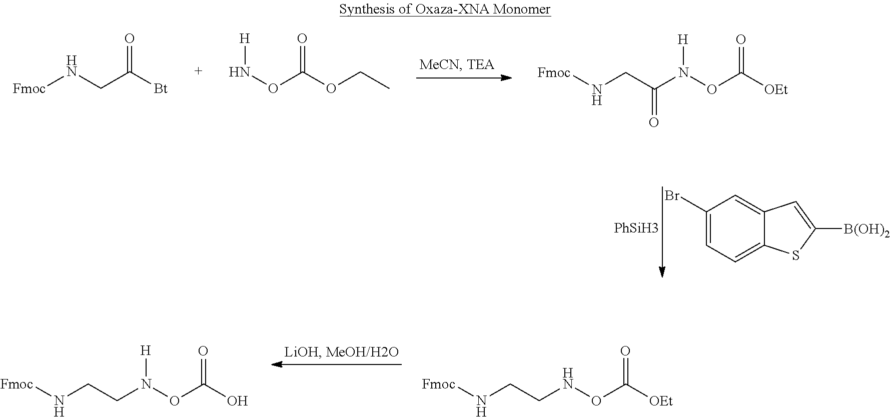

### Attachment of Protected Nucleic Acid Bases and Solid Phase Synthesis of XNA Oligomers

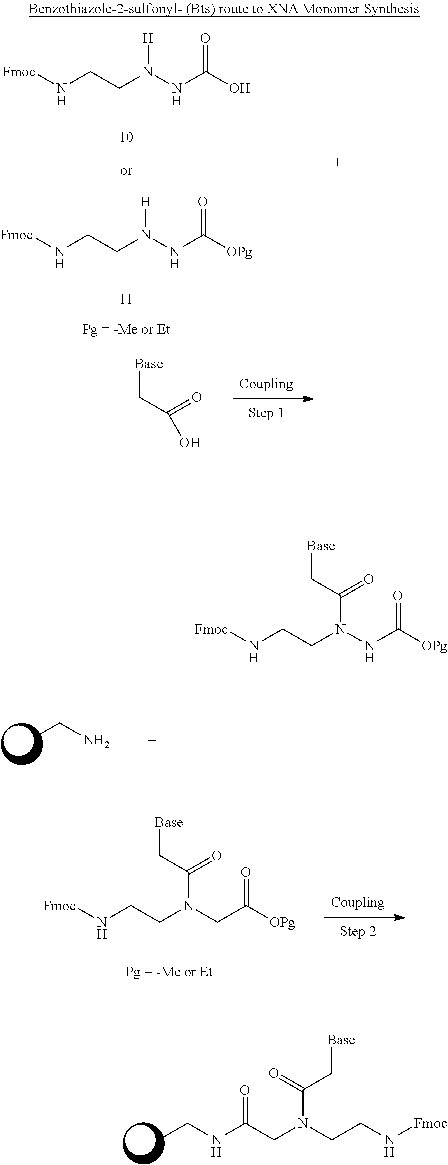

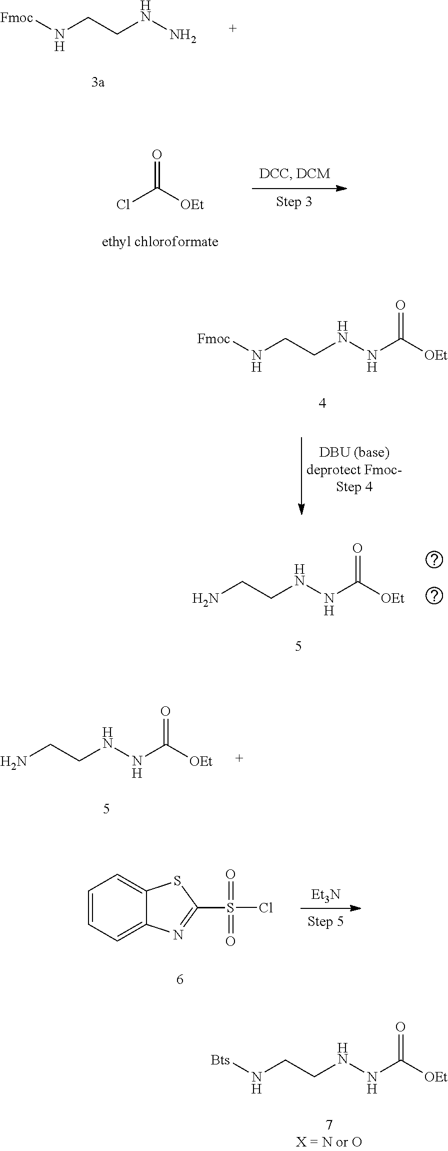

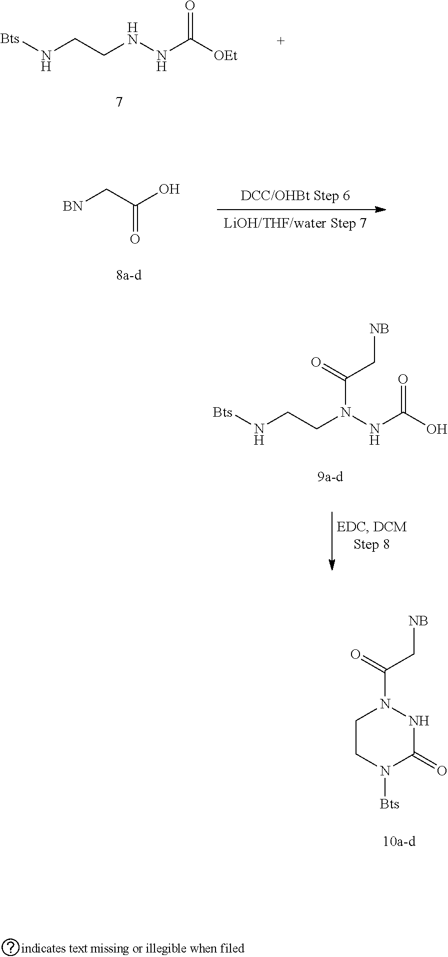

We could also introduce CDI (carbonyldiimidazole chemistry; by doing that we may skip Step 7 in above and can get to the final cyclized monomer.

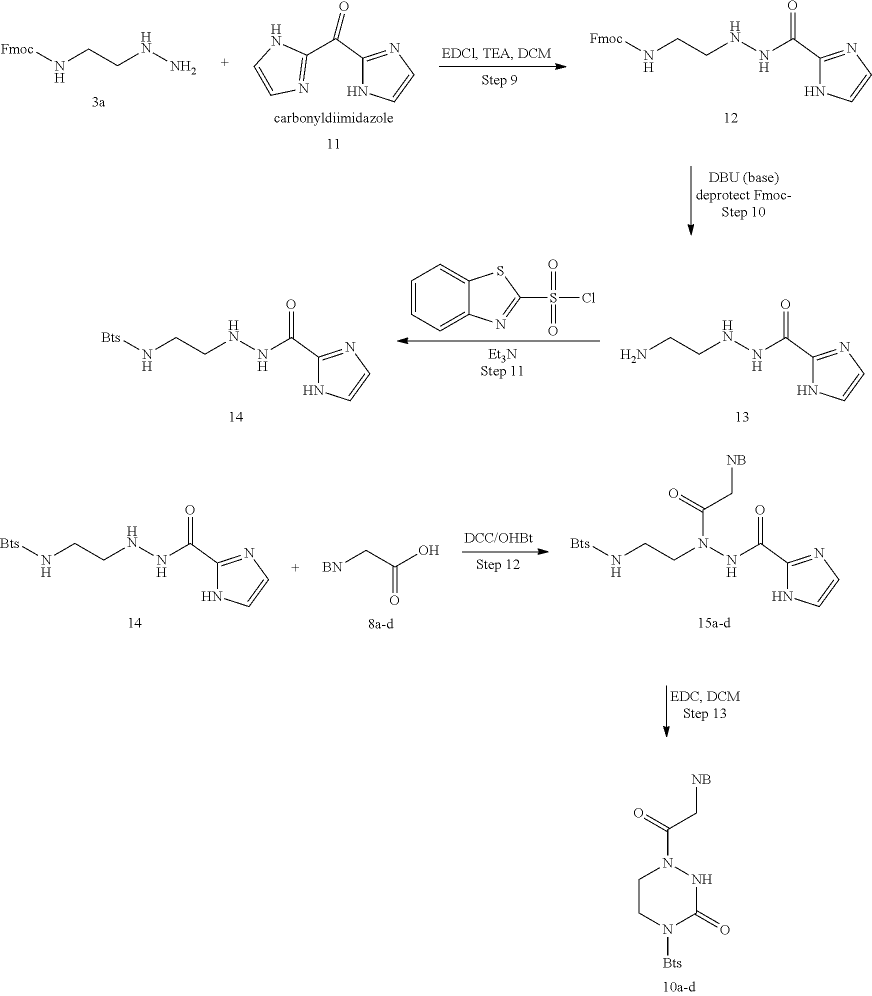

Another aza-aza compound having the structure below is made by the following synthetic steps:

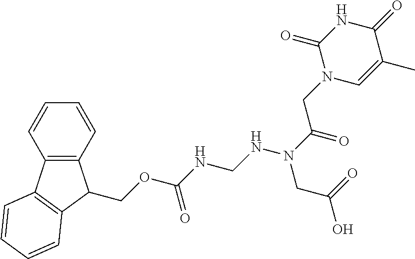 [SMILES: Cc1cn(CC(=O)N(CC(=O)O)NCNC(=O)OCC2c3ccccc3-c3ccccc32)c(=O)[nH]c1=O]

### Thymine Aza-aza Analog

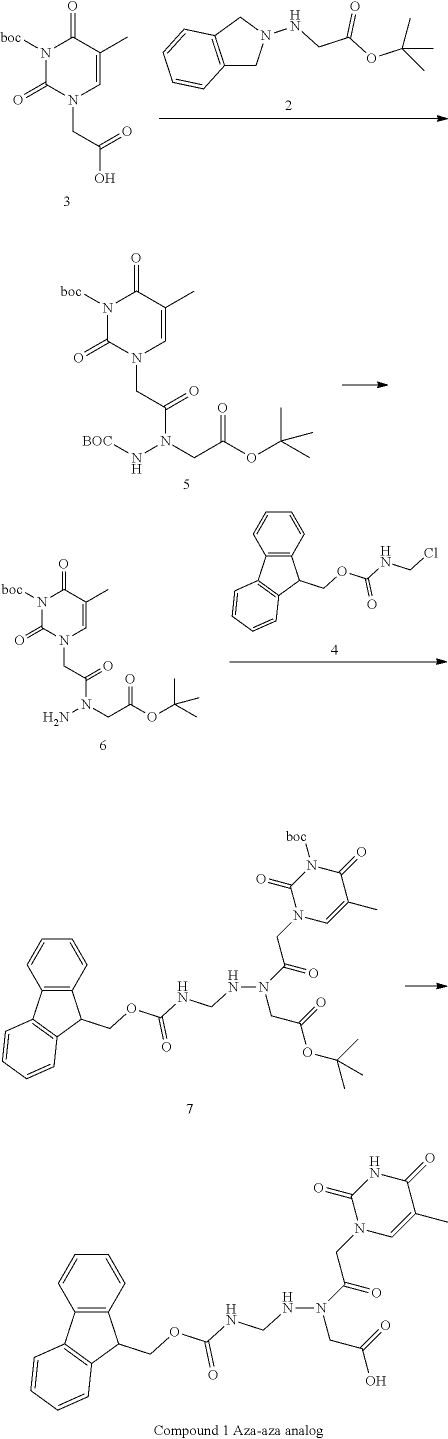

Another compound of the invention is a thio-aza compound having the following chemical structure and made by the synthetic scheme below:

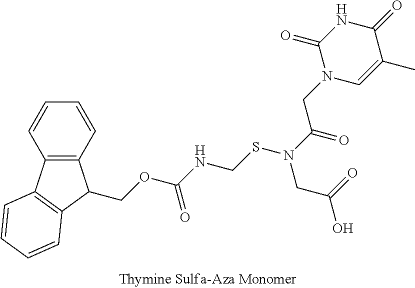 [SMILES: Cc1cn(CC(=O)N(CC(=O)O)SCNC(=O)OCC2c3ccccc3-c3ccccc32)c(=O)[nH]c1=O]

The synthesis of the above compound is as follows:

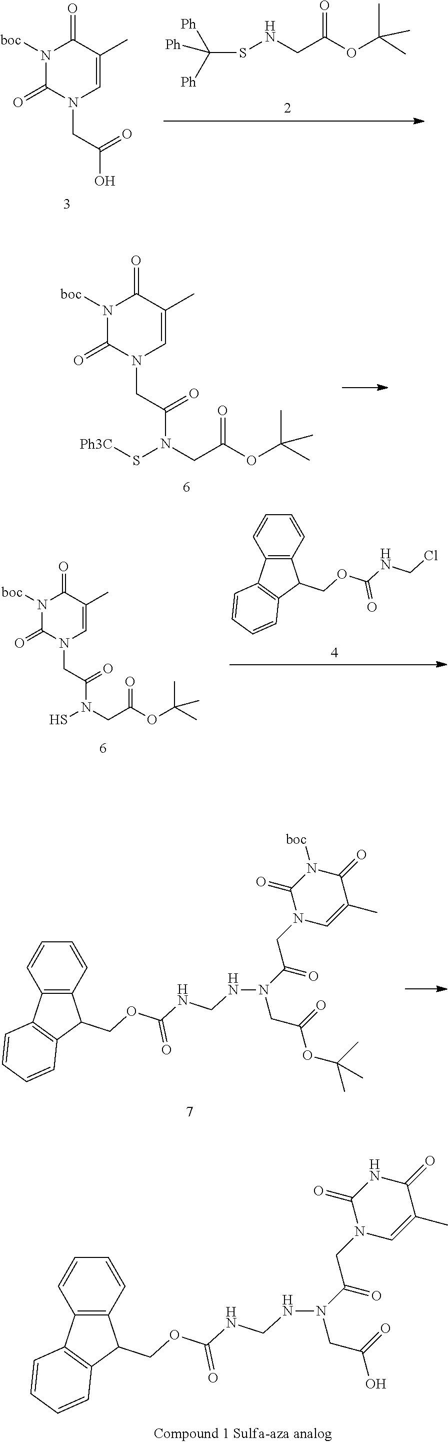

The synthetic methodology of the invention is used to synthesize the following aza-aza and oxy-aza compounds:

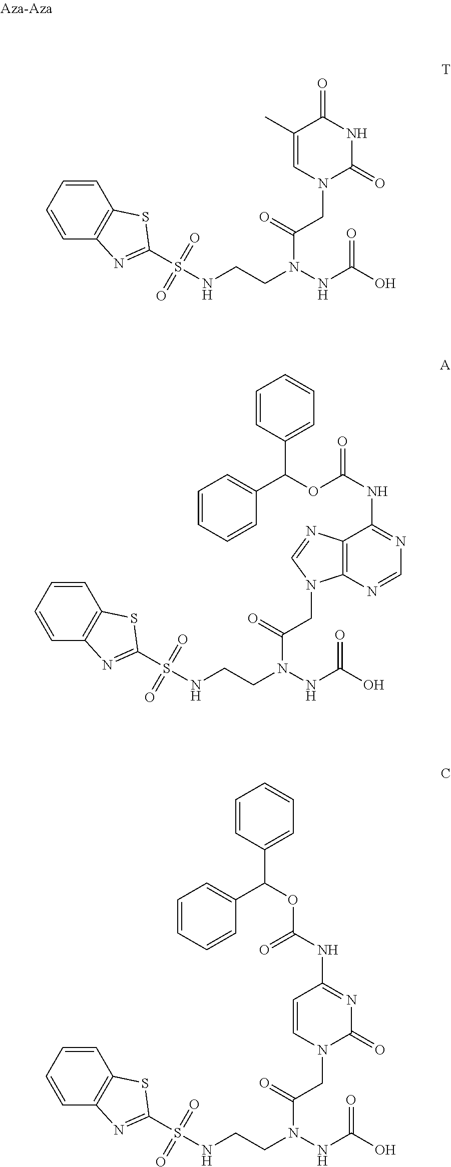 [SMILES: Cc1cn(CC(=O)N(CCNS(=O)(=O)c2nc3ccccc3s2)NC(=O)O)c(=O)[nH]c1=O ; O=C(O)NN(CCNS(=O)(=O)c1nc2ccccc2s1)C(=O)Cn1cnc2c(NC(=O)OC(c3ccccc3)c3ccccc3)ncnc21 ; O=C(O)NN(CCNS(=O)(=O)c1nc2ccccc2s1)C(=O)Cn1ccc(NC(=O)OC(c2ccccc2)c2ccccc2)nc1=O]

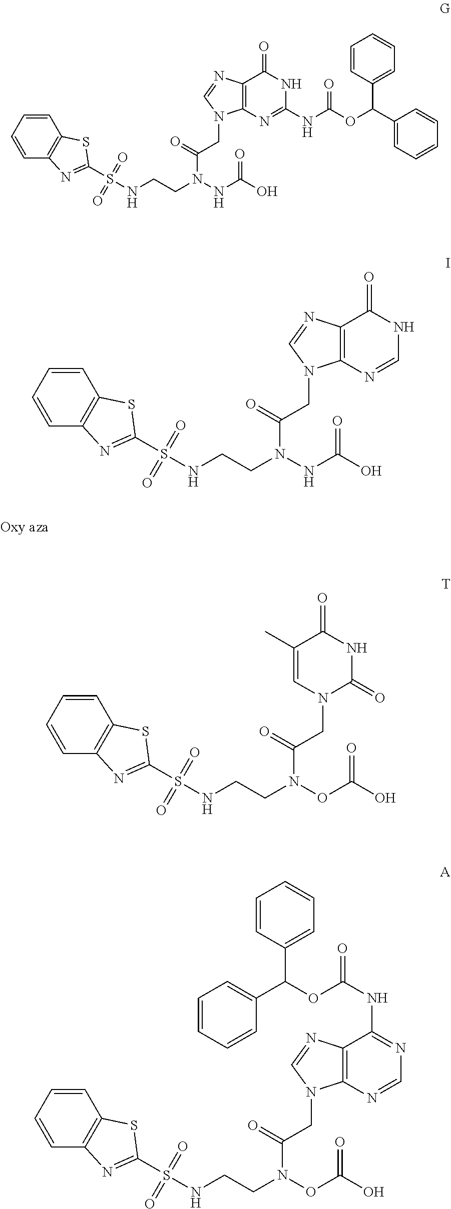 [SMILES: O=C(O)NN(CCNS(=O)(=O)c1nc2ccccc2s1)C(=O)Cn1cnc2c(=O)[nH]c(NC(=O)OC(c3ccccc3)c3ccccc3)nc21 ; O=C(O)NN(CCNS(=O)(=O)c1nc2ccccc2s1)C(=O)Cn1cnc2c(=O)[nH]cnc21 ; Cc1cn(CC(=O)N(CCNS(=O)(=O)c2nc3ccccc3s2)OC(=O)O)c(=O)[nH]c1=O ; O=C(O)ON(CCNS(=O)(=O)c1nc2ccccc2s1)C(=O)Cn1cnc2c(NC(=O)OC(c3ccccc3)c3ccccc3)ncnc21]

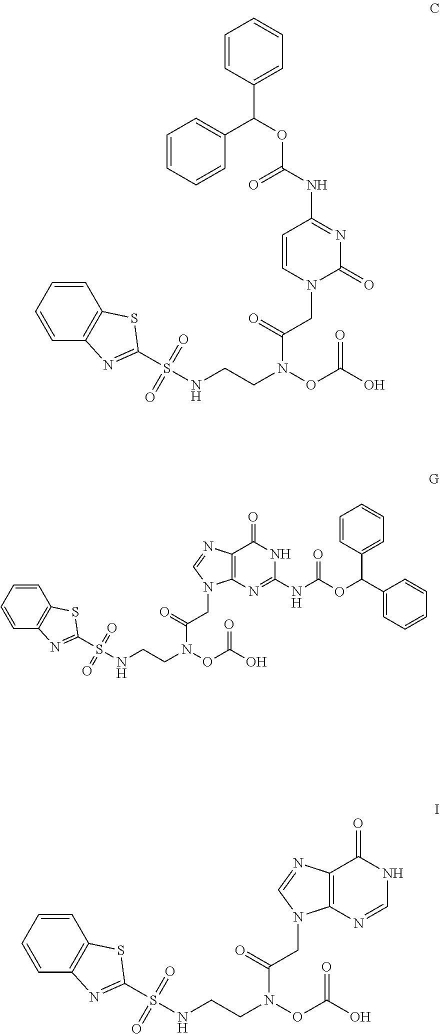 [SMILES: O=C(O)ON(CCNS(=O)(=O)c1nc2ccccc2s1)C(=O)Cn1ccc(NC(=O)OC(c2ccccc2)c2ccccc2)nc1=O ; O=C(O)ON(CCNS(=O)(=O)c1nc2ccccc2s1)C(=O)Cn1cnc2c(=O)[nH]c(NC(=O)OC(c3ccccc3)c3ccccc3)nc21 ; O=C(O)ON(CCNS(=O)(=O)c1nc2ccccc2s1)C(=O)Cn1cnc2c(=O)[nH]cnc21]

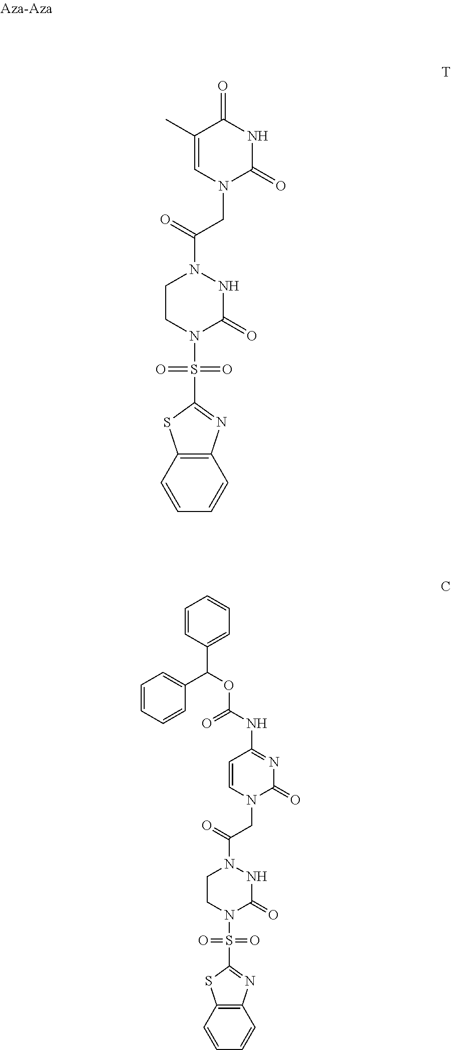 [SMILES: Cc1cn(CC(=O)N2CCN(S(=O)(=O)c3nc4ccccc4s3)C(=O)N2)c(=O)[nH]c1=O ; O=C(Nc1ccn(CC(=O)N2CCN(S(=O)(=O)c3nc4ccccc4s3)C(=O)N2)c(=O)n1)OC(c1ccccc1)c1ccccc1]

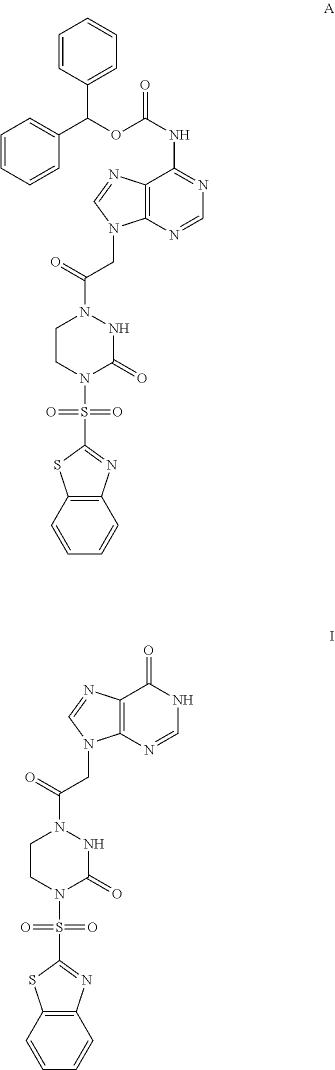 [SMILES: O=C(Nc1ncnc2c1ncn2CC(=O)N1CCN(S(=O)(=O)c2nc3ccccc3s2)C(=O)N1)OC(c1ccccc1)c1ccccc1 ; O=C(Cn1cnc2c(=O)[nH]cnc21)N1CCN(S(=O)(=O)c2nc3ccccc3s2)C(=O)N1]

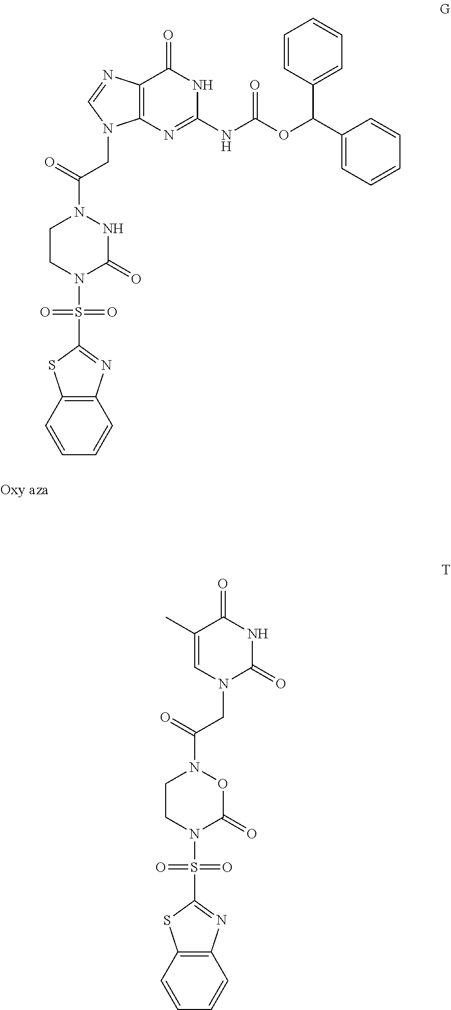 [SMILES: O=C(Nc1nc2c(ncn2CC(=O)N2CCN(S(=O)(=O)c3nc4ccccc4s3)C(=O)N2)c(=O)[nH]1)OC(c1ccccc1)c1ccccc1 ; Cc1cn(CC(=O)N2CCN(S(=O)(=O)c3nc4ccccc4s3)C(=O)O2)c(=O)[nH]c1=O]

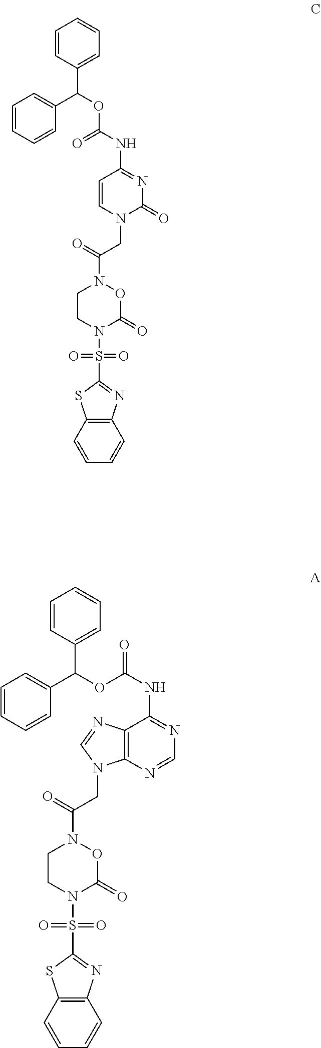 [SMILES: O=C(Nc1ccn(CC(=O)N2CCN(S(=O)(=O)c3nc4ccccc4s3)C(=O)O2)c(=O)n1)OC(c1ccccc1)c1ccccc1 ; O=C(Nc1ncnc2c1ncn2CC(=O)N1CCN(S(=O)(=O)c2nc3ccccc3s2)C(=O)O1)OC(c1ccccc1)c1ccccc1]

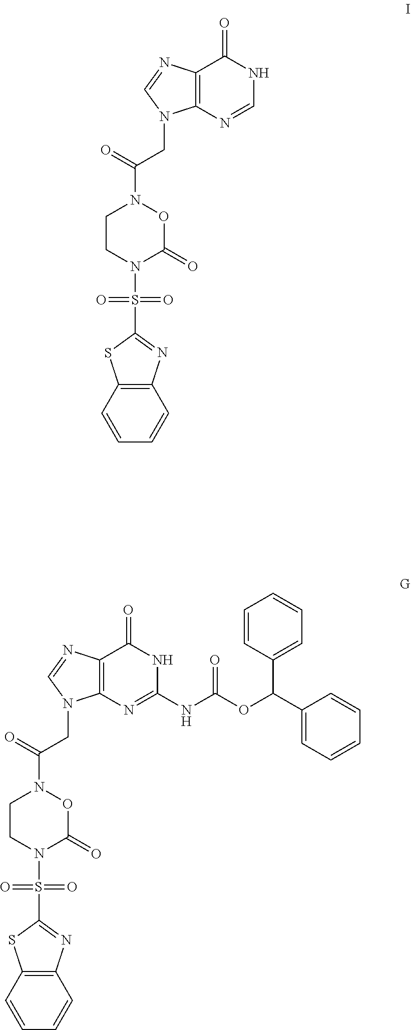 [SMILES: O=C(Cn1cnc2c(=O)[nH]cnc21)N1CCN(S(=O)(=O)c2nc3ccccc3s2)C(=O)O1 ; O=C(Nc1nc2c(ncn2CC(=O)N2CCN(S(=O)(=O)c3nc4ccccc4s3)C(=O)O2)c(=O)[nH]1)OC(c1ccccc1)c1ccccc1]

The synthetic scheme below is used to make alternative isomeric forms of aza-XNA isomer:

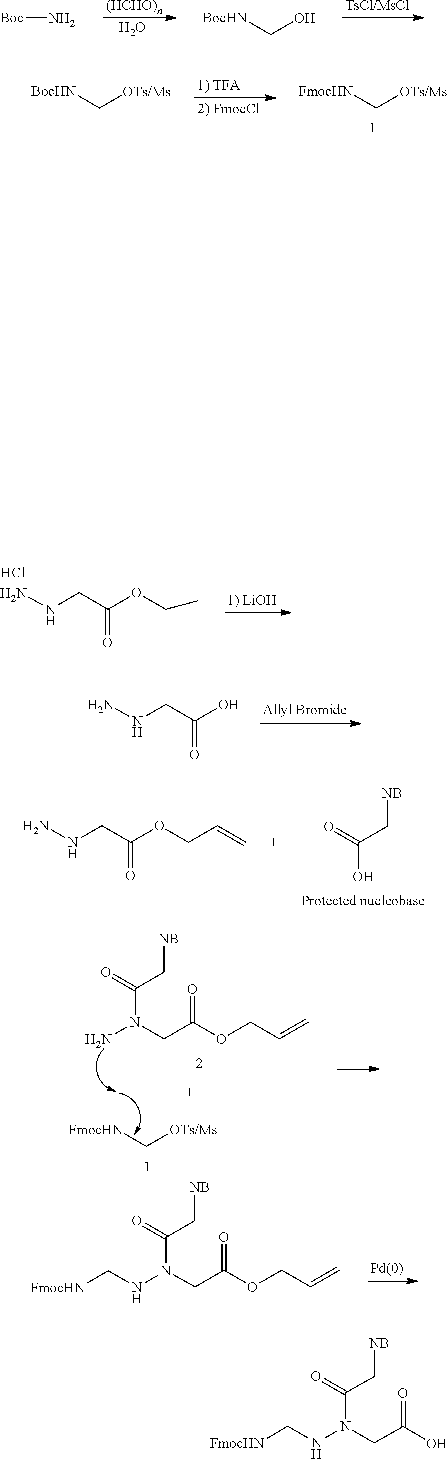

The methods disclosed herein can be used to analyze nucleic acids of samples. The term “sample” as described herein can include bodily fluids (including, but not limited to, blood, urine, feces, serum, lymph, saliva, anal and vaginal secretions, perspiration, peritoneal fluid, pleural fluid, effusions, ascites, and purulent secretions, lavage fluids, drained fluids, brush cytology specimens, biopsy tissue (e.g., tumor samples), explanted medical devices, infected catheters, pus, biofilms and semen) of virtually any organism, with mammalian samples, particularly human samples.

Amplification primers useful in the embodiments disclosed herein are preferably between 10 and 45 nucleotides in length. For example, the primers can be at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or more nucleotides in length. Primers can be provided in any suitable form, included bound to a solid support, liquid, and lyophilized, for example. In some embodiments, the primers and/or probes include oligonucleotides that hybridize to a reference nucleic acid sequence over the entire length of the oligonucleotide sequence. Such sequences can be referred to as “fully complementary” with respect to each other. Where an oligonucleotide is referred to as “substantially complementary” with respect to a nucleic acid sequence herein, the two sequences can be fully complementary, or they may form mismatches upon hybridization, but retain the ability to hybridize under stringent conditions or standard PCR conditions as discussed below. As used herein, the term “standard PCR conditions” include, for example, any of the PCR conditions disclosed herein, or known in the art, as described in, for example, PCR 1: A Practical Approach, M. J. McPherson, P. Quirke, and G. R. Taylor, Ed., (c) 2001, Oxford University Press, Oxford, England, and PCR Protocols: Current Methods and Applications, B. White, Ed., (c) 1993, Humana Press, Totowa, N.J. The amplification primers can be substantially complementary to their annealing region, comprising the specific mutant/variant target sequence(s) or the wild type target sequence(s). Accordingly, substantially complementary sequences can refer to sequences ranging in percent identity from 100, 99, 98, 97, 96, 95, 94, 93, 92, 91, 90, 89, 85, 80, 75 or less, or any number in between, compared to the reference sequence. Conditions for enhancing the stringency of amplification reactions and suitable in the embodiments disclosed herein, are well-known to those in the art. A discussion of PCR conditions, and stringency of PCR, can be found, for example in Roux, K. “Optimization and Troubleshooting in PCR,” in Pcr Primer: A Laboratory Manual, Diffenbach, Ed. ©1995, Cold Spring Harbor Laboratory Press, Cold Spring Harbor, N.Y.; and Datta, et al. (2003) Nucl. Acids Res. 31(19):5590-5597.

Provided herein are methods useful in the detection of sequence variants, i.e., insertions, deletions, nonsense mutations, missense mutations, and the like. In the methods for detecting allelic variants or variant target sequences disclosed herein, the sample, which comprises the nucleic acids to be analyzed, are contacted with an amplification primer pair, i.e., comprising a forward primer and a reverse primer that flank the target sequence or target region containing a sequence of interest {e.g., a wild-type, mutant, or variant allele sequence) to be analyzed. By “flanking” the target sequence, it is understood that the variant or wild-type allelic sequence is located between the forward and reverse primers, and that the binding site of neither the forward nor reverse primer comprises the variant or wild-type allelic sequence to be assessed. For example, in some embodiments, the variant or wild-type allelic sequence to be assessed is removed from or positioned away from the 3′ end of either oligonucleotide by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more, e.g., 100 or more, 200 or more, 300 or more, 400 or more, 500 or more, etc., nucleotides. Amplification primers that flank, but that do not overlap with, the variant target sequence or the wild-type target sequence are thus not “allele-specific” amplification primers, and are capable of amplification of various different alleles or variants of a sequence of interest. Thus, in some embodiments, the amplification primers are configured to amplify various mutant or variant alleles and wild type alleles non-preferentially. As discussed in further detail below, the addition of XNA to an amplification reaction suppresses the amplification of wild-type target sequences and enables preferential amplification of non-wild-type, e.g., variant, mutant or rare variant alleles. FIG. 1 is a depictions of exemplary method according to the embodiments disclosed herein for the detection of sequence variants. As shown in FIG. 1, amplification primers (i.e., forward primer 1 and reverse primer 2) flank the wild type and mutant allele sequences of interest, and comprise sequences common to both wild-type and mutant or variant allele sequences. Accordingly, as shown in FIG. 1, in contrast to methods that utilize allele-specific amplification primers to achieve preferential amplification of rare sequences, the present methods advantageously enable the simultaneous amplification of multiple variant sequences, using a single amplification primer pair.

In a second embodiment, the invention relates to compositions and multiplex methods for the detection of genetic variations (mutations) in DNA templates derived from lung cancer biological samples with xeno-nucleic acid clamping probes. The first method employs multi-color fluorescence detection using locus specific fluorescent hybridization probes (Hyb Probes), hydrolysis (TaqMan or ZEN) probes or molecular beacons. The second method employs mutant specific amplicon capture probes immobilized on multiple bar-coded capture beads.

Current XNA clamping qPCR methodologies utilize a single tube-single mutation detection format, it is preferable to detect multiple genetic variations in a single tube thus reducing the complexity of the assay and the amount of template DNA required for analysis.

This second embodiment of the invention is directed to the use of locus specific fluorescent probes designed to detect the genetic variant (mutant) amplicons generated during the XNA clamping PCR reaction. This second embodiment discloses locus specific probes that bind to mutant specific amplicons at a region upstream or downstream from the site of the mutation to be detected. Furthermore, the second embodiment discloses the use of multiplexed XNA clamping qPCR reactions that are able to detect multiple mutations (at least 6 or more) in one PCR reaction tube using fluorescence detection methodology.

In a third embodiment of the invention, there is provided a method the rapid isolation of genetic material present in circulating cells and also cell-free genetic material from biological fluids and the determination of genetic variations in those cells and biological fluids. Such biological fluids include: blood, serum, plasma, saliva, mucus, urine, sputum, semen or other biological secretions. In this embodiment, the invention also provides the detection of somatic and germ-line mutations in the genetic material derived from these biological fluids utilizing gene amplification and xeno-nucleic acid clamping.

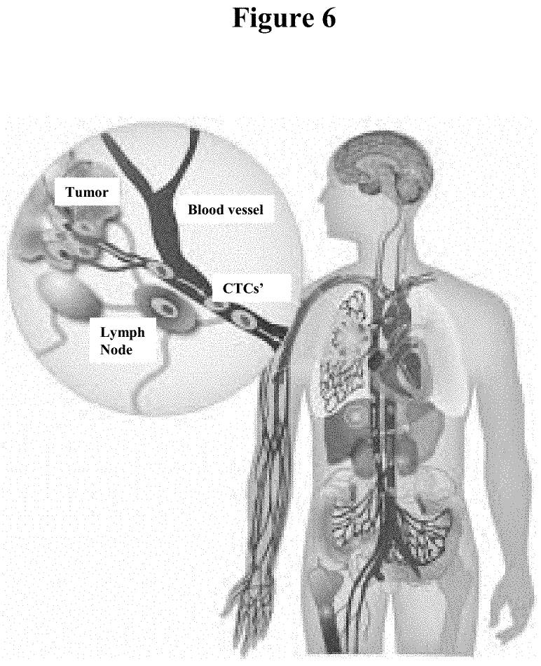

Circulating tumor cells (CTC's) and cell-free DNA (cfDNA) derived from tumor cells are present in the peripheral blood of cancer patients (See FIG. 6). Tumor derived DNA can also be found in the urine and even the saliva of cancer patients.

In general, circulating free DNA is smaller in size than DNA derived directly from a surgical biopsy or FFPE sample. This embodiment also describes a novel sample treatment procedure that utilizes a novel lysis reagent called QZol™. QZol™ sample lysis is a direct one tube procedure and an aliquot of the lysate is used directly in molecular genetic and cytogenetic analysis procedures such as PCR, RTPCR, FISH, Next Generation Sequencing (NGS) and branched DNA (bDNA) assays. The QZol™ procedure eliminates the tedious multistep preanalytical processing that is currently used in Molecular Pathology and Cytogenetic analysis.

The lysis reagent is a 50% solution (A) containing chaotropic salts and detergent (nonionic, anionic, cationic or zwitterionic) and a 50% solution (B) containing neutralizing reagents and stabilizers.

This invention also concerns to the specific amplification of genetic variant templates from the isolated genetic material described above. Only target genetic material that has a variation, e.g. single nucleotide polymorphism (SNP), gene deletion or insertion and/or translocation or truncation is amplified in a quantitative primer directed polymerase chain reaction (qPCR). This is achieved utilising xenonucleic acid (XNA) probe clamping sequences that have been designed to bind specifically by Watson-Crick base pairing to wild-type sequences in the sample. The presence of the XNA probes in the qPCR primer mix employed for the target amplification reaction causes inhibition of the polymerase mediated amplification of wild-type templates but does not impede the amplification of mutant template sequences.

The methods disclosed herein can be used in the detection of numerous allelic variants, including nonsense mutations, missense mutations, insertions, deletions, and the like. Owing to the advantageous sensitivity and specificity of detection afforded by the methods disclosed herein, the methods can detect the presence of a rare allelic variant within a sample, amongst a high wild-type background. Accordingly, although the skilled artisan will appreciate that the methods disclosed herein can be used in a variety of settings to detect, e.g., germline mutations, the methods are particularly well-suited for use in the detection of somatic mutations, such as mutations present in tumors.

Non-limiting examples of rare, somatic mutations useful in the diagnosis, prognosis, and treatment of various tumors include, for example, the genetic mutations/variations that are in a gene selected from the group consisting of: EGFR gene (codons 858, 746, 790, 861, 719, 768, and 769), KRAS gene (codons 12 and 61), NRAS gene (codons 12 and 61), PIK3CA (codons 542 and 1047), and BRAF (codon 600) associated with the non-small cell lung cancer (NSCLC).

The practice of the present subject matter may employ, unless otherwise indicated, conventional techniques and descriptions of organic chemistry, molecular biology (including recombinant techniques), cell biology, and biochemistry, which are within the skill of the art. Such conventional techniques include, but are not limited to, preparation of synthetic polynucleotides, polymerization techniques, chemical and physical analysis of polymer particles, preparation of nucleic acid libraries, nucleic acid sequencing and analysis, and the like. Specific illustrations of suitable techniques can be used by reference to the examples provided herein. Other equivalent conventional procedures can also be used. Such conventional techniques and descriptions can be found in standard laboratory manuals such as Genome Analysis: A Laboratory Manual Series (Vols. I-IV), PCR Primer: A Laboratory Manual, and Molecular Cloning: A Laboratory Manual (all from Cold Spring Harbor Laboratory Press), Hermanson, Bioconjugate Techniques, Second Edition (Academic Press, 2008); Merkus, Particle Size Measurements (Springer, 2009); Rubinstein and Colby, Polymer Physics (Oxford University Press, 2003); and the like.

In the preferred embodiment of the invention, a Lung Cancer Multiplex Taqman Assay is provided for qualitative detection of mutations in the EGFR gene (codons 858, 746, 790, 861, 719, 768, and 769), KRAS gene (codons 12 and 61), NRAS gene (codons 12 and 61), PIK3CA (codons 542 and 1047), and BRAF (codon 600) associated with the non-small cell lung cancer (NSCLC).

The Lung Cancer Multiplex qPCR Test is designed as a real-time qPCR-based in vitro diagnostic test intended for use in the qualitative and indiscriminatory detection of the most common somatic variants in the EGFR, KRAS, NRAS, PIK3CA and BRAF gene in FFPE and plasma samples from human patients with signs and symptoms of NSCLC.

Since clinical samples from cancer patients frequently contain trace amounts of mutant allele in a large excess of wild-type DNA, XNA-PCR technology is employed in the Lung Cancer Multiplex Taqman assays to suppress amplification of WT alleles to improve the sensitivity of mutation detection.

The assay includes a set of oligonucleotide primers and dual-labeled hydrolysis (TaqManÒ) probes for use in real-time PCR assays for in vitro qualitative detection of EGFR, KRAS, NRAS, PIK3CA and BRAF gene mutations from the DNA extracted from FFPE tissue sections, frozen tissue, blood and plasma of human patients with signs and symptoms of NSCLC.

The target mutations in the EGFR, KRAS, NRAS, PIK3CA AND BRAF gene were selected based on their mutation frequency in lung cancer patients in the COSMIC database, their actionability per literature as well as the mutation coverage of several currently available on the market EGFR, KRAS, NRAS, PIK3CA AND BRAF mutation detection assays. A housekeeping gene, beta-actin (ACTβ), was selected as internal control. ACTβ assay is used to monitor sample DNA extraction efficiency and presence of PCR inhibitors as well as to provide a way of quantitation of amplifiable template in each reaction well to prevent false positive/negative results.

The primers and probes were designed using Primer3 software following the qPCR primer and probe design rules. The primers were designed with a Tm of 58-63° C. while the probes were designed with Tm of 63-68° C. The amplicon sizes were designed under 150 bp whenever possible.

Primers and probes were checked in-silico for specificity (BLAST), primer dimers/secondary structure (autoDimer) and amplicon secondary structure (M-fold).

The XNAs of the invention were designed to be between the forward and reverse primers or partially overlap with the forward primer.The probes were designed to be on the same strand as and be adjacent to the respective XNA.

Primer, probe and XNA combinations and concentration optimization tests were performed to find the optimal conditions for differentiating the mutant and the WT alleles for each targeted somatic mutation.

For efficient clamping by the XNA, an XNA annealing step at 70° C. before the binding of primers and probes was included in the qPCR cycling program. Optimal annealing temperature for primers and probes were tested by gradient analysis.

All the somatic mutations included in the Lung Cancer Multiplex Taqman assay were based on the data from literature, COSMIC and My Cancer Genome databases and the NCCN Guidelines. The sequences for EGFR, KRAS, NRAS, PIK3CA AND BRAF somatic variant DNA sequences were retrieved from the human aligned gDNA sequences database on the UCSC Genome Browser. The initial multiplexing and further optimization of the assay conditions were performed on the reference DNA templates from cell lines.

### EXAMPLES

The Instruments, reagents, software and databases used in the experiments of the invention were:
Roche LC480II DC2035, Cat. No. 05015243001
ABI 7500 Fast Dx Real-Time PCR, Cat. No. 4406985
ABI QuantStudio 5, Cat. No A28140
BioRad CFX384, Cat No
   Reagents/Kits
SensiFAST™ Lyo-Ready No-ROX Mix (Cat. #: BIO-11061)
   Software
Primer3 and Autodimer
   Browser based tools
UCSC Genome Browser
NCBI
dbSNP
dbVar
COSMIC

### Materials and Methods

### DNA Isolation

Human genomic DNA must be extracted from tissue or blood, or fixed paraffin-embedded tissue prior to use. Several methods exist for DNA isolation. For consistency, we recommend using a commercial kit, such as Qiagen DNA extraction kit (QIAamp DNA FFPE Tissue Kit, cat No. 56404, for paraffin embedded specimens; DNeasy Blood & Tissue kit, cat. No. 69504 or 69506, for tissue and blood specimens). Follow the genomic DNA isolation procedure according to manufacturer's protocol. Sufficient amounts of DNA can be isolated from FFPE blocks or fresh frozen sections (approx. 2-10 μg).

### Example 1

The compositions of the PCR reaction mixes are described in Table 1.

TABLE 1
PCR reaction mix

| | Volume in 10 μl | Final |
|---|---|---|
| Reagent | reaction, μl | concentration |
| 2x Master Mix | 5 | 1x |
| 5, 10 μm primer F | 0.2 | 100-400 nM |
| 5, 10 μm primer R | 0.2 | 100-400 nM |
| 2.5 μm primer F ACTB | 0.2 | 100 nM |
| 2.5 μm primer R ACTB | 0.2 | 100 nM |
| 2.5 μm probe ACTB | 0.1 | 100 nM |
| 10 μm probe (target) | 0.1 | 100 nM |
| Template | 2 | |
| XNA (.0625, .1875, .25, | 1 | .0625, .1875, .25, .25, |
| .25, .25, 2, 2.5 μM) | | .25, 2, 2.5 μM |
| Nuclease free water | 1 | |
| TOTAL | 10 | |

Reference templates: gBlocks were designed and ordered from IDT.

TABLE 2
QClamp gBlock sequences

| QClamp PC | QClamp PC | QClamp PC | QClamp PC |
|---|---|---|---|
| Panel 1 | Panel 2 | Panel 3 | Panel 4 |
| EGFR T790M | EGFR S768I | EGFR Ex2OinsASV | EGFR C797S |
| EGFR L858R | PIK3CA H1047R | KRAS Q61H | PIK3CA E545K |
| NRAS Q61K | NRAS G12D | EGFR G719S | BRAF V600E |
| ACTB | PIK3CA E542K | KRAS K117N | KRAS A146T |
| NRAS A146T | NRAS A59T | NRAS K117N | EGFR Exl9del |
| KRAS A59T | EGFR L861Q | KRAS Gl2D | |

QClamp PC Panel 1
SEQ ID NO: 1
ACGTATTTTGAAACTCAAGATCGCATTCATGCGTCTTCACCTGGAAGGGGTCCATGT
GCCCCTCCTTCTGGCCACCATGCGAAGCCACACTGACGTGCCTCTCCCTCCCTCCAG
GAAGCCTACGTGATGGCCAGCGTGGACAACCCCCACGTGTGCCGCCTGCTGGGCAT
CTGCCTCACCTCCACCGTGCAGCTCATCATGCAGCTCATGCCCTTCGGCTGCCTCCTG
GACTATGTCCGGGAACACAAAGACAATATTGGCTCCCAGTACCTGCTCAACTGGTGT
GTGCAGATCGCAAAGGTAATCAGGGAAGGGAGATACGGGGAGGGGAGATAAGGAG
CCAGGATCCTCACATGCGGTCTGCGCTCCTGGGATAGCAAGAGTTTGCCATGGGGAT
ATGGAGGCTCAGAGCCTGGCATGAACATGACCCTGAATTCGGATGCAGAGCTTCTTC
CCATGATGATCTGTCCCTCACAGCAGGGTCTTCTCTGTTTCAGGGCATGAACTACTTG
GAGGACCGTCGCTTGGTGCACCGCGACCTGGCAGCCAGGAACGTACTGGTGAAAAC
ACCGCAGCATGTCAAGATCACAGATTTTGGGCGGGCCAAACTGCTGGGTGCGGAAG
AGAAAGAATACCATGCAGAAGGAGGCAAAGTAAGGAGGTGGCTTTAGGTCAGCCA
GCATTTTCCTGACACCAGGGACCAGGCTGCCTTCCCACTAGCTGTATTGTTTAACAC
ATGCAGGGGAGGATGCTCTCCAGACATTCTGGGTGAGCTCGCAGCAGCTGCTGCTG
GCAGCTGGTCTATCTTCCCTAGTGTGGTAACCTCATTTCCCCATAAAGATTCAGAAC
ACAAAGATCATCCTTTCAGAGAAAATAATGCTCCTAGTACCTGTAGAGGTTAATATC
CGCAAATGACTTGCTATTATTGATGGCAAATACACAGAGGAAGCCTTCGCCTGTCCT
CATGTATTGGTCTCTCATGGCACTGTACTCTTCTTTTCCAGCTGTATCCAGTATGTCC
AACAAACAGGTTTCACCATCTATAACCACTTGTTTTCTGTAAGAATCCTGGGGGTGT
GGAGGGTAAGGGGGCAGGGAGGGAGGGAAGTTCAATTTTTATTAAAAACCACAGG
GAATGCAATGCTATTGCCAAGGTTAAATAAGCATCTAACTATTCAAGCCCATTTCTG
CCTATCTGGTGCTCGGTGAGGATCTTCATGAGGTAGTCAGTCAGGTCCCGGCCAGCC
AGGTCCAGACGCAGGATGGCATGGGGGAGGGCATACCCCTCGTAGATGGGCACAGT
GTGGGTGACCCCGTCACCGGAGTCCATCACGATGCCAGTGGTACGGCCAGAGGCGT
ACAGGGATAGCACAGCCTGGATAGCAACGTACATGGCTGGGGTGTTGAAGGTCTCA
AACATGATCTGTAAGGCAGAGATACACCATGTCACACTGGGGAAGCCACTGGGGAC
AGCCAGGCCAGACGGGGGACATGCAGAAAGTGCAAAGAACACGGCTAAGTGTGCT
GGGGTCTTGGGATGGGGAGTCTGTTCAGACCTACTGTGCACCTACTTAATACACACT
CCAAGGCCGCTTTACACCTGAGATCACACCACTGCACTCCAGCTTAGAAGATAGAGT
GAGACTCTGCCTAAAAAAAAATAAAAAATAAAAAAATAAAAATGAAAAAAATGCA
TAACAACAAAGAATATGAATATGGATCACATCTCTACCAGAGTTAATCAACTGATGC
AAACTCTTGCACAAATGCTGAAAGCTGTACCATACCTGTCTGGTCTTGGTTGAGGTT
TCAATGAATGGAATCCCGTAACTCTTGGCCAGTTCGTGGGCTTGTTTTGTATCAACTG
TCCTTGTTGGCAAATCACACTTGTTTCCCACTAGCACCATAGGTACATCATCCGAGTC
TTTTACTCGCTTAATCTGCTCCCTAAAAACGGGAATATATTATCAGAACATAAGAAA
AACAAGATTAGGCTGGGTTATATGCATGGCATTAGCAAAGACTCAAAAAATAAAAA
CTATAATTACTCCTTAATGTCAGCTTATTATATTCAATTTAAACCCACCTATAATGGT
GAATATCTTCAAATGATTTAGTATTATTTATGGCAAATACACAAAGAAAGCCCTCCC
CAGTCCTCATGTACTGGTCCCTCATTGCACTGTACTCCTCTTGACCTGTTGTGTCGAG
AATATCCAAGAGACAGGTTTCTCCATCAATTACTACTTGCTTCCTGTAGGAATCCTG
AGAAGGGAGAAACACAGTCTGGATTATTACAGTGCACCTTTTACTTCAAAAAAGGT
GTTATATACAACTCAACAACAAAAAATTCAATTTAAAAATGGGCAAAGGACTTGAA
AAGACATTGTTCCTGCTCCA
QClamp PC Panel 2
SEQ ID NO: 2
ACTTCACAGCCCTGCGTAAACGTCCCTGTGCTAGGTCTTTTGCAGGCACAGCTTTTCC
TCCATGAGTACGTATTTTGAAACTCAAGATCGCATTCATGCGTCTTCACCTGGAAGG
GGTCCATGTGCCCCTCCTTCTGGCCACCATGCGAAGCCACACTGACGTGCCTCTCCC
TCCCTCCAGGAAGCCTACGTGATGGCCATCGTGGACAACCCCCACGTGTGCCGCCTG
CTGGGCATCTGCCTCACCTCCACCGTGCAGCTCATCACGCAGCTCATGCCCTTCGGC
TGCCTCCTGGACTATGTCCGGGAACACAAAGACAATATTGGCTCCCAGTACCTGCTC
AACTGGTGTGTGCAGATCGCAAAGGTAATCAGGGAAGGGAGATACGGGGAGGGGA
GAGGAGATGTGTTACAAGGCTTATCTAGCTATTCGACAGCATGCCAATCTCTTCATA
AATCTTTTCTCAATGATGCTTGGCTCTGGAATGCCAGAACTACAATCTTTTGATGACA
TTGCATACATTCGAAAGACCCTAGCCTTAGATAAAACTGAGCAAGAGGCTTTGGAGT
ATTTCATGAAACAAATGAATGATGCACGTCATGGTGGCTGGACAACAAAAATGGAT
TGGATCTTCCACACAATTAAACAGCATGCATTGAACTGAAAAGATAACTGAGAAAA
TGAAAGCTCACTCTGGATTCCACACTGCACTGTTAATAACTCTCAGCAGGCAAAGAC
CGATTGCATAGGAATTGCACAATCCATGAACAGCATTAGAATTTACAGCAAGAACA
GAAATATGTATACCCAAAATAACTTTTTACTTTCTCTCCTCTTATTCCTTTAATACAG
AATATGGGTAAAGATGATCCGACAAGTGAGAGACAGGATCAGGTCAGCGGGCTACC
ACTGGGCCTCACCTCTATGGTGGGATCATATTCATCTACAAAGTGGTTCTGGATTAG
CTGGATTGTCAGTGCGCTTTTCCCAACACCATCTGCTCCAACCACCACCAGTTTGTAC
TCAGTCATTTCACACCAGCAAGAACCTGTTGGAAACCAGTAATCAGGGTTAATTGGC
GAGCCACATCTACAGTACTTTAAAGCTTTCTATAATCAATGGAAATGAAAACCCTAG
TGTGACCTTCCATTTGGTTCTTAAAGTGACTAGAGAACGCAAAAACACCGGATTAAT
ATCGGAGCAATGTAAAATTTATTGAAAATGTATTTGCTTTTTCTGTAAATCATCTGTG
AATCCAGAGGGGAAAAATATGACAAAGAAAGCTATATAAGATATTATTTTATTTTAC
AGAGTAACAGACTAGCTAGAGACAATGAATTAAGGGAAAATGACAAAGAACAGCT
CAAAGCAATTTCTACACGAGATCCTCTCTCTAAAATCACTGAGCAGGAGAAAGATTT
TCTATGGAGTCACAGGTAAGTGCTAAAATGGAGATTCTCTGTTTCTTTTTCTTTATTA
CAGAAAAAATAACTGAATTTGGCTGATCTCAGCATGTTTTTACCATACCTATTGGAA
TAAATAAAGCAGAATTTACATGATTTTTAAACTATAAACATTGCCTTTTTAAAAACA
ATGGTTATCTTCCCTAGTGTGGTAACCTCATTTCCCCATAAAGATTCAGAACACAAA
GATCATCCTTTCAGAGAAAATAATGCTCCTAGTACCTGTAGAGGTTAATATCCGCAA
ATGACTTGCTATTATTGATGGCAAATACACAGAGGAAGCCTTCGCCTGTCCTCATGT
ATTGGTCTCTCATGGCACTGTACTCTTCTTGTCCAGTTGTATCCAGTATGTCCAACAA
ACAGGTTTCACCATCTATAACCACTTGTTTTCTGTAAGAATCCTGGGGGTGTGGAGG
GTAAGGGGGCAGGGAGGGAGGGAAGTTCAATTTTTATTAAAAACCACAGGGAATGC
AATGCTATTGCCAAGGTTAAATAAGCATCTAACTATTCAAGCCCATTTCTGCCTATCT
GGTTTGAGAGCCTGGCATGAACATGACCCTGAATTCGGATGCAGAGCTTCTTCCCAT
GATGATCTGTCCCTCACAGCAGGGTCTTCTCTGTTTCAGGGCATGAACTACTTGGAG
GACCGTCGCTTGGTGCACCGCGACCTGGCAGCCAGGAACGTACTGGTGAAAACACC
GCAGCATGTCAAGATCACAGATTTTGGGCTGGCCAAACAGCTGGGTGCGGAAGAGA
AAGAATACCATGCAGAAGGAGGCAAAGTAAGGAGGTGGCTTTAGGTCAGCCAGCAT
TTTCCTGACACCAGGGACCAGGCTGCCTTCCCACTAGCTGTATTGTTTAACACATGC
AGGGGAGGATGCTCTCCAGACATTCTGGGTGAGCTCGCAGCAGCTGCTGCTGGCAG
CTGGGTCCAGCACTTCACAGCCCTGCGTAAACGTCCCTGTGCTAGGTCTTTTGCAGG
CACAGCTTTTCCTCCATGAGTACGTATTTTGAAACTCAAGATCGCATTCATGCGTCTT
CACCTGGAAGGGGTCCATGTGCCCCTCCTTCTGGCCACCATGCGAAGCCACACTGAC
GTGCCTCTCCCTCCCTCCAGGAAGCCTACGTGATGGCCATCGTGGACAACCCCCACG
TGTGCCGCCTGCTGGGCATCTGCCTCACCTCCACCGTGCAGCTCATCACGCAGCTCA
TGCCCTTCGGCTGCCTCCTGGACTATGTCCGGGAACACAAAGACAATATTGGCTCCC
AGTACCTGCTCAACTGGTGTGTGCAGATCGCAAAGGTAATCAGGGAAGGGAGATAC
GGGGAGGGGAGAGGAGATGTGTTACAAGGCTTATCTAGCTATTCGACAGCATGCCA
ATCTCTTCATAAATCTTTTCTCAATGATGCTTGGCTCTGGAATGCCAGAACTACAATC
TTTTGATGACATTGCATACATTCGAAAGACCCTAGCCTTAGATAAAACTGAGCAAGA
GGCTTTGGAGTATTTCATGAAACAAATGAATGATGCACGTCATGGTGGCTGGACAAC
AAAAATGGATTGGATCTTCCACACAATTAAACAGCATGCATTGAACTGAAAAGATA
ACTGAGAAAATGAAAGCTCACTCTGGATTCCACACTGCACTGTTAATAACTCTCAGC
AGGCAAAGACCGATTGCATAGGAATTGCACAATCCATGAACAGCATTAGAATTTAC
AGCAAGAACAGAAATATGTATACCCAAAATAACTTTTTACTTTCTCTCCTCTTATTCC
TTTAATACAGAATATGGGTAAAGATGATCCGACAAGTGAGAGACAGGATCAGGTCA
GCGGGCTACCACTGGGCCTCACCTCTATGGTGGGATCATATTCATCTACAAAGTGGT
TCTGGATTAGCTGGATTGTCAGTGCGCTTTTCCCAACACCATCTGCTCCAACCACCA
CCAGTTTGTACTCAGTCATTTCACACCAGCAAGAACCTGTTGGAAACCAGTAATCAG
GGTTAATTGGCGAGCCACATCTACAGTACTTTAAAGCTTTCTATAATCAATGGAAAT
GAAAACCCTAGTGTGACCTTCCATTTGGTTCTTAAAGTGACTAGAGAACGCAAAAAC
ACCGGATTAATATCGGAGCAATGTAAAATTTATTGAAAATGTATTTGCTTTTTCTGTA
AATCATCTGTGAATCCAGAGGGGAAAAATATGACAAAGAAAGCTATATAAGATATT
ATTTTATTTTACAGAGTAACAGACTAGCTAGAGACAATGAATTAAGGGAAAATGAC
AAAGAACAGCTCAAAGCAATTTCTACACGAGATCCTCTCTCTAAAATCACTGAGCAG
GAGAAAGATTTTCTATGGAGTCACAGGTAAGTGCTAAAATGGAGATTCTCTGTTTCT
TTTTCTTTATTACAGAAAAAATAACTGAATTTGGCTGATCTCAGCATGTTTTTACCAT
ACCTATTGGAATAAATAAAGCAGAATTTACATGATTTTTAAACTATAAACATTGCCT
TTTTAAAAACAATGGTTATCTTCCCTAGTGTGGTAACCTCATTTCCCCATAAAGATTC
AGAACACAAAGATCATCCTTTCAGAGAAAATAATGCTCCTAGTACCTGTAGAGGTTA
ATATCCGCAAATGACTTGCTATTATTGATGGCAAATACACAGAGGAAGCCTTCGCCT
GTCCTCATGTATTGGTCTCTCATGGCACTGTACTCTTCTTGTCCAGTTGTATCCAGTA
TGTCCAACAAACAGGTTTCACCATCTATAACCACTTGTTTTCTGTAAGAATCCTGGG
GGTGTGGAGGGTAAGGGGGCAGGGAGGGAGGGAAGTTCAATTTTTATTAAAAACCA
CAGGGAATGCAATGCTATTGCCAAGGTTAAATAAGCATCTAACTATTCAAGCCCATT
TCTGCCTATCTGGTTTGAGAGCCTGGCATGAACATGACCCTGAATTCGGATGCAGAG
CTTCTTCCCATGATGATCTGTCCCTCACAGCAGGGTCTTCTCTGTTTCAGGGCATGAA
CTACTTGGAGGACCGTCGCTTGGTGCACCGCGACCTGGCAGCCAGGAACGTACTGG
TGAAAACACCGCAGCATGTCAAGATCACAGATTTTGGGCTGGCCAAACAGCTGGGT
GCGGAAGAGAAAGAATACCATGCAGAAGGAGGCAAAGTAAGGAGGTGGCTTTAGG
TCAGCCAGCATTTTCCTGACACCAGGGACCAGGCTGCCTTCCCACTAGCTGTATTGT
TTAACACATGCAGGGGAGGATGCTCTCCAGACATTCTGGGTGAGCTCGCAGCAGCT
GCTGCTGGCAGCTGGGTCCAGC
QClamp PC Panel 3
SEQ ID NO: 3
AGCCCTGCGTAAACGTCCCTGTGCTAGGTCTTTTGCAGGCACAGCTTTTCCTCCATGA
GTACGTATTTTGAAACTCAAGATCGCATTCATGCGTCTTCACCTGGAAGGGGTCCAT
GTGCCCCTCCTTCTGGCCACCATGCGAAGCCACACTGACGTGCCTCTCCCTCCCTCC
AGGAAGCCTACGTGATGGCCAGCGCCAGCGTGGTGGACAACCCCCACGTGTGCCGC
CTGCTGGGCATCTGCCTCACCTCCACCGTGCAGCTCATCACGCAGCTCATGCCCTTC
GGCTGCCTCCTGGACTATGTCCGGGAACACAAAGACAATATTGGCTCCCAGTACCTG
CTCAACTGGTGTGTGCAGATCGCAAAGGTAATCAGGGAAGGGAGATACGGGGAGGG
GAATATGCATGGCATTAGCAAAGACTCAAAAAATAAAAACTATAATTACTCCTTAAT
GTCAGCTTATTATATTCAATTTAAACCCACCTATAATGGTGAATATCTTCAAATGATT
TAGTATTATTTATGGCAAATACACAAAGAAAGCCCTCCCCAGTCCTCATGTACTGGT
CCCTCATTGCACTGTACTCCTCGTGACCTGCTGTGTCGAGAATATCCAAGAGACAGG
TTTCTCCATCAATTACTACTTGCTTCCTGTAGGAATCCTGAGAAGGGAGAAACACAG
TCTGGATTATTACAGTGCACCTTTTACTTCAAAAAAGGTGTTATATACAACTCAACA
ACAAAAAATTCAATTTAAAAATGGGCAAAGGACTTGAAAAGACATTGTTCCTGCTC
CAAGCCCATGCCGTGGCTGCTGGTCCCCCTGCTGGGCCATGTCTGGCACTGCTTTCC
AGCATGGTGAGGGCTGAGGTGACCCTTGTCTCTGTGTTCTTGTCCCCCCCAGCTTGTG
GAGCCTCTTACACCCAGTGGAGAAGCTCCCAACCAAGCTCTCTTGAGGATCTTGAAG
GAAACTGAATTCAAAAAGATCAAAGTGCTGAGCTCCGGTGCGTTCGGCACGGTGTA
TAAGGTAAGGTCCCTGGCACAGGCCTCTGGGCTGGGCCGCAGGGCCTCTCATGGTCT
GGTGGGGAGCCCAGAGTCCTTGCAAGCTGTATATTTCCATCATCTACTTTACTCTTTG
TTTCACTGAGTGTTTGGGAAACTCCAGTGTTTTTCCCAAGTTATTGAGAGGAAATCTT
TTTTCCTAGTATAGCATAATTGAGAGAAAAACTGATATATTAAATGACATAACAGTT
ATGATTTTGCAGAAAACAGATCTGTATTTATTTCAGTGTTACTTACCTGTCTTGTCTT
TGCTGATGTTTCAATAAAAGGAATTCCATAACTTCTTGCTAAGTCCTGAGCCTGTTTT
GTGTCTACTGTTCTAGAAGGCAAATCACAGTTATTTCCTACTAGGACCATAGGTACA
TCTTCAGAGTCCTTAACTCTTTTAATTTGTTCTCTGGGAAAGAAAAAAAAGTTATAG
CACAGTCATTAGTAACACAAATATCTTTCAAAACCTGTCCACAACTTTTGTCATAAA
ATTTGGCTGAAAGAAAACAATGTAATTCCTAGTTTCCACTACACCAAATTTTCCTTCC
AAAATGCATAACAACAAAGAATATGAATATGGATCACATCTCTACCAGAGTTAATC
AACTGATGCAAACTCTTGCACAAATGCTGAAAGCTGTACCATACCTGTCTGGTCTTG
GCTGAGGTTTCAATGAATGGAATCCCGTAACTCTTGGCCAGTTCGTGGGCTTGTTTT
GTATCAACTGTCCTTGTTGGCAAATCACAGTTGTTTCCCACTAGCACCATAGGTACA
TCATCCGAGTCTTTTACTCGCTTAATCTGCTCCCTAAAAACGGGAATATATTATCAGA
ACATAAGAAAAACAAGATTAGGCTGGGTACAGTGGCTCATGCCTGCAATCTCAGCA
CTTTGGGAGGCTGAGGAGGGCAGACTGCTTGAACCCAGGAGTTTGAGATTAGCCTG
GGCCATTTCAGATAACTTAACTTTCAGCATAATTATCTTGTAATAAGTACTCATGAA
AATGGTCAGAGAAACCTTTATCTGTATCAAAGAATGGTCCTGCACCAGTAATATGCA
TATTAAAACAAGATTTACCTCTATTGTTGGATCATATTCGTCCACAAAATGATTCTGA
ATTAGCTGTATCGTCAAGGCACTCTTGCCTACGCCATCAGCTCCAACTACCACAAGT
TTATATTCAGTCATTTTCAGCAGGCCTTATAATAAAAATAATGAAAATGTGACTATA
TTAGAACATGTCACACATAAGGTTAATACACTATCAAATACTCCACCAGTACCTTTT
AATACAAACTCACCTTTATATGAAAAATTATTTCAAAATACCTTACAAAATTCAATC
ATG
QClamp PC Panel 4
SEQ ID NO: 4
AGATCGCATTCATGCGTCTTCACCTGGAAGGGGTCCATGTGCCCCTCCTTCTGGCCA
CCATGCGAAGCCACACTGACGTGCCTCTCCCTCCCTCCAGGAAGCCTACGTGATGGC
CAGCGTGGACAACCCCCACGTGTGCCGCCTGCTGGGCATCTGCCTCACCTCCACCGT
GCAGCTCATCACGCAGCTCATGCCCTTCGGCTCCCTCCTGGACTATGTCCGGGAACA
CAAAGACAATATTGGCTCCCAGTACCTGCTCAACTGGTGTGTGCAGATCGCAAAGGT
AATCAGGGAAGGGAGATACGGGGAGGGGAGATAAGGAGCCAGGATCCTCACATGC
GGTCTGCGCTCCTGGGATAGCAAGAGTTTGCCATGGGGATATGTGTGTGCGTGCATG
CAGCACACACACAAAATTTATTGAAAATGTATTTGCTTTTTCTGTAAATCATCTGTGA
ATC CAGAGGGGAAAAATATGACAAAGAAAGCTATATAAGATATTATTTTATTTTACA
GAGTAACAGACTAGCTAGAGACAATGAATTAAGGGAAAATGACAAAGAACAGCTC
AAAGCAATTTCTACACGAGATCCTCTCTCTGAAATCACTAAGCAGGAGAAAGATTTT
CTATGGAGTCACAGGTAAGTGCTAAAATGGAGATTCTCTGTTTCTTTTTCTTTATTAC
AGAAAAAATAACTGAATTTGGCTGATCTCAGCATGTTTTTACCATACCTATTGGAAT
AAATAAAGCAGAATTTACATGATTTTTAAACTATAAACATTGCCTTTTTAAAAACAA
TGGTTGTAAATTGAAAAAAATAAGAACACTGATTTTTGTGAATACTGGGAACTATGA
AAATACTATAGTTGAGACCTTCAATGACTTTCTAGTAACTCAGCAGCATCTCAGGGC
CAAAAATTTAATCAGTGGAAAAATAGCCTCAATTCTTACCATCCACAAAATGGATCC
AGACAACTGTTCAAACTGATGGGACCCACTCCATCGAGATTTCTCTGTAGCTAGACC
AAAATCACCTATTTTTACTGTGAGGTCTTCATGAAGAAATATATCTGAGGTGTAGTA
AGTAAAGGAAAACAGTAGATCTCATTTTCCTATCAGAGCAAGCATTATGAAGAGTTT
AGGTAAGAGATCTAATTTCTATAATTCTGTAATATAATATTCTTTAAAACATAGTACT
TCATCTTTCCTCTTAACCAAAGCCAAAAGCAGTACCATGGACACTGGATTAAGAAGC
AATGCCCTCTCAAGAGACAAAAACATTTACTAAATATTGTTTTATTTCCTAGTATAG
CATAATTGAGAGAAAAACTGATATATTAAATGACATAACAGTTATGATTTTGCAGAA
AACAGATCTGTATTTATTTCAGTGTTACTTACCTGTCTTGTCTTTGTTGATGTTTCAAT
AAAAGGAATTCCATAACTTCTTGCTAAGTCCTGAGCCTGTTTTGTGTCTACTGTTCTA
GAAGGCAAATCACATTTATTTCCTACTAGGACCATAGGTACATCTTCAGAGTCCTTA
ACTCTTTTAATTTGTTCTCTGGGAAAGAAAAAAAAGTTATAGCACAGTCATTAGTAA
CACAAATATCTTTATCAGCCTTAGGTGCGGCTCCACAGCCCCAGTGTCCCTCACCTT
CGGGGTGCATCGCTGGTAACATCCACCCAGATCACTGGGCAGCATGTGGCACCATCT
CACAATTGCCAGTTAACGTCTTCCTTCTCTCTCTGTCATAGGGACTCTGGATCCCAGA
AGGTGAGAAAGTTAAAATTCCCGTCGCTATCAAGACATCTCCGAAAGCCAACAAGG
AAATCCTCGATGTGAGTTTCTGCTTTGCTGTGTGGGGGTCCATGGCTCTGAACCTCA
GGCCCACCTTTTCTCATGTCTGGCAGCTGCTCTGCTCTAGACCCTGCTCATCTCCACA
TCCTAAATGTTCACTTTCTATGTCTTTCCCTTTCTAGCTCTAGTGGGTATAACT

### Example 2

### Feasibility Testing and Master Mix

Based on previous tests on master mixes for Taqman based qPCR assays, SensiFAST™ Lyo-Ready No-ROX Mix was selected as the primary master mix for Taqman probe-based qPCR reactions for mutation detection assay development.

### Example 3

### Primers, Probes and XNA Sequences and Optimal Final Concentrations

Several primer pairs and probes were designed and tested for each of the targets in single gene target assays. The primers and probes that displayed the earliest Cts were selected for the assay development. The primers selected for the Lung Cancer Multiplex Taqman assays are in Table 3.

TABLE 3

| Assay | Forward Primer | Reverse Primer |
|---|---|---|
| EGFR L858 | EGFR L858/861 F2 | EGFR L861 R2 |
| EGFR E746-A750 Del | EGFR Ex19del F2 | EGFR Ex19del R2 |
| EGFR T790M | EGFR T790M Geno F2 | EGFR T790 R2 |
| EGFR L861 | EGFR L861 F4 | EGFR L861 R2 |
| EGFR G719 | EGFR G719 F2 | EGFR G719 R2 |
| EGFR S768i | EGFR S768i Geno F5 | EGFR S768i/Ex20 ins R5 |
| EGFR V769_D770 ins | EGFR V770 ins Gen F | EGFR S768i/Ex20 ins R5 |
| EGFR C797S | EGFR C797SF2 | EGFR C797S R1 |
| KRAS G12D | KRAS G12/13-F3 | KRAS G12/13-R3 |
| KRAS Q61H | KRASc61F004 | KRASc61R004 |
| NRAS G12D | NRAS G12/13-F2 | NRAS G12/13-R2 |
| NRAS Q61K | NRAS Q61-F3 | NRASA59/Q61-R2 |
| PIK3CA E542K | PIK3CA E542 F | PIK3CA E542 R |
| PIK3CA H1047R | PIK3CA H1047 F | PIK3CA H1047 R |
| BRAF V600 | BRAF V600E F2 | BRAF V600E* F2 |
| | BRAF V600K F2 | BRAF V600R F2 |
| | BRAF V600 R1 | |
| Reference Assay ACTB | ACTB-F3 Primer | ACTB-R2 Primer |

The Sequences of the Lung Cancer Multiplex assay primers are described in Table 4.

TABLE 4

| | | | Final Primer |
|---|---|---|---|
| | | | Concentration |
| Target | Primer Name | Primer Sequence | (nM) |
| EGFR L858 | EGFR L858/861 F2 | SEQ ID NO: 5 TGCCTCCTTCTGCATGGTAT | 100 |
| EGFR L858 | EGFR L858 R2 | SEQ ID NO: 6 AGCCAGGAACGTACTGGTGA | 100 |
| EGFR T790M | EGFR T790M Geno F2 | SEQ ID NO: 7 CACCGTGCAGCTCATCAT | 100 |
| EGFR T790M | EGFR T790 R2 | SEQ ID NO: 8 GTCTTTGTGTTCCCGGACAT | 100 |
| EGFR L861 | EGFR L861 F4 | SEQ ID NO: 9 CTTTCTCTTCCGCACCCA | 100 |
| EGFR L861 | EGFR L861 R2 | SEQ ID NO: 10 AGCCAGGAACGTACTGGTGA | 100 |
| EGFR G719 | EGFR G719 F2 | SEQ ID NO: 11 CCTTATACACCGTGCCGAAC | 100 |
| EGFR G719 | EGFR G719 R2 | SEQ ID NO: 12 TGGAGCCTCTTACACCCAGT | 100 |
| EGFR S768i | EGFR S768i Geno F5 | SEQ ID NO: 13 GGGGGTTGTCCACGAT | 200 |
| EGFR S768i | EGFR S768i/Ex20 ins | SEQ ID NO: 14 ACCATGCGAAGCCACACT | 200 |
| EGFR | EGFR V770 ins Geno F | SEQ ID NO: 15 CACGCTGGCCACGCT | 400 |
| V769_D770 ins | | | |
| EGFR | EGFR S768i/Ex20 ins | SEQ ID NO: 16 ACCATGCGAAGCCACACT | 400 |
| V769_D770 | R5 | | |
| EGFR C797S | EGFR C7975 F2 | SEQ ID NO: 17 GCTCATGCCCTTCGGC | 200 |
| EGFR C797S | EGFR C7975 R1 | SEQ ID NO: 18 GCAGGTACTGGGAGCCAAT | 200 |
| KRAS G12D | KRAS G12/G13 - F3 | SEQ ID NO: 19 AAGATTTACCTCTATTGTTGGAT | 200 |
| | | CATATTC | |
| KRAS G12D | KRAS G12/G13 - R3 | SEQ ID NO: 20 TGCTGAAAATGACTGAATATAA | 200 |
| | | ACTTGT | |
| KRAS Q61H | KRASc61F004 | SEQ ID NO: 21 CCCTCATTGCACTGTACTCCTC | 100 |
| KRAS Q61H | KRASc61R004 | SEQ ID NO: 22 CCAGACTGTGTTTCTCCCTTCT | 100 |
| NRAS G12D | NRAS G12/13 - F2 | SEQ ID NO: 23 CTCACCTCTATGGTGGGATCA | 200 |
| NRAS G12D | NRAS G12/13 - R2 | SEQ ID NO: 24 CAGGTTCTTGCTGGTGTGAA | 200 |
| NRAS Q61K | NRAS Q61 F3 | SEQ ID NO: 25 GTCTCTCATGGCACTGTACTCTTC | 400 |
| NRAS Q61K | NRAS A59/Q61 R2 | SEQ ID NO: 26 CACCCCCAGGATTCTTACAG | 400 |
| PIK3CA E542K | PIK3CA E542 F | SEQ ID NO: 27 TCAGAGAAGCCATTATCTGCAA | 400 |
| PIK3CA E542K | PIK3CA E542 R | SEQ ID NO: 28 ACTCCATAGAAAATCTTTCTCCT | 400 |
| | | GCT | |
| PIK3CA H1047R | PIK3CA H1047 F | SEQ ID NO: 29 GCAAGAGGCTTTGGAGTATTTCA | 400 |
| PIK3CA H1047R | PIK3CA H1047 R | SEQ ID NO: 30 GAAGATCCAATCCATTTTTGTTG | 400 |
| | | TC | |
| BRAF V600 | BRAF V600E F2 | SEQ ID NO: 31 GGTGATTTTGGTCTAGCTACGGA | 100 |
| BRAF V600 | BRAF V600F* F2 | SEQ ID NO: 32 TGATTTTGGTCTAGCTACGGAA | 100 |
| BRAF V600 | BRAF V600K F2 | SEQ ID NO: 33 AGTGATTTTGGTCTAGCTACGAA | 100 |
| BRAF V600 | BRAF V600R F2 | SEQ ID NO: 34 AGTGATTTTGGTCTAGCTACGAG | 100 |
| BRAF V600 | BRAF V600 R1 | SEQ ID NO: 35 CATCCACAAAATGGATCCAGACAA | 100 |
| ACTB | ACTB - F3 | SEQ ID NO: 36 TCTGCCTTACAGATCATGTTTGAG | 100 |
| ACTB | ACTB - R2 | SEQ ID NO: 37 CCAGAGGCGTACAGGGATAG | 100 |

The Sequences of the Lung Cancer Multiplex Taqman assay probes are described in Table 5.

TABLE 5

| | | | Final Probe |
|---|---|---|---|
| | | | Concentration |
| Target | Probe Name | Probe Sequence | (nM) |
| EGFR L858R | EGFR L858r Geno Pr1 | SEQ ID NO: 38 TTTGGCCGGCCCAAAAT | 100 |
| | FAM | CTG | |
| EGFR E746- | EGFR Exl9del Pr2 - TEX | SEQ ID NO: 39 GCGACGGGAATTTTAAC | 100 |
| A750 | | TTTCTCAC | |
| EGFR T790M | EGFR T790M Pr2 FAM | SEQ ID NO: 40 GCTCAGCACGCAGCTCA | 100 |
| | | TGC | |
| EGFR L861Q | EGFR L861 Pr2 - TEX | SEQ ID NO: 41 TGTGATCTTGACATGCTG | 100 |
| | | CGG | |
| EGFR 719A | EGFR G719 Pr2 - HEX | SEQ ID NO: 42 CAGTTTCCTTCAAGATCC | 100 |
| and S | | TCAAGAGA | |
| EGFR S768i | S768i/Ex20 ins Pr5 - HEX | SEQ ID NO: 43 TGGAGGGAGGGAGAGG | 100 |
| | | CAC | |
| EGFR | S768i/Ex20 ins Geno Pr5 - | SEQ ID NO: 44 TGGAGGGAGGGAGAGG | 100 |
| V769_D770 ins | TEX | CAC | |
| EGFR C797S | EGFR C797S Pr. 1 FAM | SEQ ID NO: 45 CTATGTCCGGGAACACA | 200 |
| | | AAGACAAT | |
| KRAS G12D | KRAS G12/G13 Pr. 2 - | SEQ ID NO: 46 TGAATTAGCTGTATCGT | 100 |
| | HEX | CAAGGCAC | |
| KRAS Q61H | KRAS A59/Q61 Pr2 - TEX | SEQ ID NO: 47 CCAAGAGACAGGTTTCT | 100 |
| | | CCATCAATTAC | |
| NRAS G12D | NRAS G12/G13 Pr2 FAM | SEQ ID NO: 48 /56-FAM/AGTGGTTCT/ | 200 |
| | | ZEN/GGATTAGCTGGATTGTC/3IABkFQ/ | |
| NRAS Q61K | NRAS A59/Q61 Pr2 - HEX | SEQ ID NO: 49 /56-FAM/CAAACAGGT/ | 400 |
| | | ZEN/TTCACCATCTATAACCACTT/3IABkFQ/ | |
| PIK3CA | PIK3CA E542 Pr FAM | SEQ ID NO: 50 ACGAGATCCTCTCTCTA | 200 |
| E542K | | AAATCACTG | |
| PIK3CA | PIK3CA H1047 Pr - HEX | SEQ ID NO: 51 TGAATGATGCACGTCATG | 200 |
| H1047R | | GTGGCTG | |
| BRAF V600 | BRAF600Pr01 - TEX | SEQ ID NO: 52 CAAACTGATGGGACCCA | 100 |
| | | CTCCATCG | |
| ACTB | ACTB-Pr2-Cy5 | SEQ ID NO: 53 CCATGTACGTTGCTATC | 100 |
| | | CAGGCTGT | |

The Sequences of the Lung Cancer Multiplex Taqman assay XNAs are described in Table 6.

TABLE 6

| | | | Final XNA |
|---|---|---|---|
| | | | Concentrations |
| Target | XNA Name | XNA Sequence | (nM) |
| EGFR L858R | DPCE004D | SEQ ID NO: 54 D-LYS-O-GGCCAGCCCAAA | 90 |
| | | ATCTGT | |
| EGFR L861Q | DPCE005B | SEQ ID NO: 55 D-LYS-O-ACCCAGCAGTTTG | 250 |
| | | GC | |
| EGFR E746- | DPCE002C | SEQ ID NO: 56 D-LYS-O-CGGAGATGTTGCT | 187.5 |
| A750 del. | | TCTCTTAATTCC | 187.5 |
| EGFR T790M | DPCE008G | SEQ ID NO: 57 Ac-TCATCACGCAGCTC-NH2 | 140 |
| EGFR G719A | DPCE001B | SEQ ID NO: 58 D-LYS-O-CGGAGCCCAGCAC | 187.5 |
| and S | | TTTGAT | |
| EGFR 768i | Blocker | SEQ ID NO: 59 TTGTCCACGCTGGCCAT/3SpC3/ | 2000 |
| EGFR | Blocker | SEQ ID NO: 60 TTGTCCACGCTGGCCAT/3SpC3/ | 2500 |
| V769_D770 ins | | | |
| EGFR c797S | 08 (PNA2) | SEQ ID NO: 61 TTCGGCTGCCTCCTGG | 1500 |
| KRAS G12 | DPCK001C2 | SEQ ID NO: 62 D-Lys-O-CTACGCCACCAGCT | 1000 |
| | | CCAACTACCA | |
| KRAS Q61 | DPCK003B | SEQ ID NO: 63 D-Lys-O-TGTACTCCTCTTGAC | 500 |
| | | CTGCTGTG | |
| NRAS G12 | DPCN001B | SEQ ID NO: 64 D-Lys-O-CAACACCACCTGCT | 375 |
| | | CCAACCACCACNH2 | |
| NRAS Q61 | DPCN003C | SEQ ID NO: 65 D-Lys-O-GGCACTGTACTCTT | 500 |
| | | CTTGTCCAGNH2 | |
| PIK3CA c542 | DPCKA003B | SEQ ID NO: 66 D-LYS-O-AGATCCTCTCTCT | 1000 |
| | | GAAATCAC | |
| PIK3CA H1047 | DPCKA005 | SEQ ID NO: 67 D-LYS-O-AATGATGCACATCA | 62.5 |
| | | TGGTGGCTG | |
| BRAF V600 | DPCBR001B | SEQ ID NO: 68 D-Lys-O-ATCGAGATTTCACT | 750 |
| | | GTAGCTAGAC | |

The primer, probe and and XNA final concentrations of the Lung Cancer Multiplex Taqman assays are described in Table 7.

TABLE 7

| | Best 1X | Best 1X | Best 1X |
|---|---|---|---|
| | Primer | Probe | XNA |
| | Concentration | Concentrations | Concentrations |
| Assay | (nM) | (nM) | (nM) |
| EGFR Ex19 del | 100 | 100 | 187.5 |
| EGFR T790M | 100 | 100 | 140 |
| EGFR G719S | 100 | 100 | 187.5 |
| EGFR L861Q | 100 | 100 | 250 |
| EGFR C797S | 200 | 200 | 1500 |
| KRAS Gl2D | 200 | 100 | 1000 |
| EGFR V769_D770ins | 400 | 100 | 2500 |
| EGFR L858R | 100 | 100 | 90 |
| PIK3CA H1047R | 400 | 200 | 62.5 |
| KRAS Q61H | 100 | 100 | 500 |
| PIK3CA E542K | 400 | 200 | 1000 |
| EGFR S768I | 200 | 100 | 2000 |
| BRAF V600 | 100 | 100 | 750 |
| NRAS Gl2D | 200 | 200 | 375 |
| NRAS Q61K | 400 | 400 | 500 |

### Example 4

### Panel Combinations

The Lung Cancer Multiplex QClamp contains 15 targets in 5 panels in the following layout as shown in Table 8. In addition to the targets, each panel has an internal control ACTB assay (IC) with a probe labelled Cy5.

TABLE 8
Composition of Lung Cancer Multiplex QClamp panels

| Panel | Target 1 (FAM) | Target 2 (Hex) | Target 3 (Tex) |
|---|---|---|---|
| A | EGFR T790M | EGFR c719 | EGFR Ex19 del |
| B | EGFR cC797S | KRAS c12 | EGFR c861 |
| C | EGFR L858R | PIK3CA c1047 | EGFR Ex20ins ASV |
| D | KRAS c61 | EGFR S768I | PIK3CA c542 |
| E | NRAS c61 | BRAF c600 | NRAS c12 |

Thresholds for different qPCR instruments are shown in Table 9

TABLE 9
THRESHOLDS

| | | | | ABI 7500 |
|---|---|---|---|---|
| | QS5 | BioRad | LC480 | Fast DX |
| FAM | 25000 | 200 | 0.8 | 30000 |
| HEX | 10000 | 200 | 0.7 | 30000 |
| TEX | 10000 | 350 | 0.5 | 30000 |
| Cy5 | 10000 | 250 | 0.5 | 30000 |

Preliminary limits of detection on different qPCR instruments.

### Example 5

Preliminary limits of detection (LOD) of the Lung Cancer Multiplex QClamp assay in ABI QuantStudio 5. LOD dCq are in grey background. The data in Table 10 shows that panel A-E's LOD was 0.13%-0.5% VAF in ABI QS5 instrument.

### Example 6

Preliminary limits of detection (LOD) of the Lung Cancer Multiplex QClamp assay in Roche LC480II. LOD dCq are in grey background. The data in Table 11 shows that panel A-E's LOD was 0.13%-0.5% VAF in LC480II instrument.

### Example 7

Preliminary limits of detection (LOD) of the Lung Cancer Multiplex QClamp assay in BioRad CFX384. LOD dCq are in grey background. The data in Table 12 shows that panel A-E's LOD was 0.13%-0.5% VAF in CFX384 instrument.

### Example 8

Preliminary limits of detection (LOD) of the Lung Cancer Multiplex QClamp assay in ABI 7500 Fast Dx. LOD dCq are in grey background. The data in Table 13 shows that panel A-E's LOD was 0.13%-0.5% VAF in ABI 7500 Fast Dx instrument.

### Example 9

Table 14 shows the normalization of gBlock concentration vs gDNA

TABLE 14

| | | Ave | Ave | PC | CC | Ave | Ave | PC | CC | Ave | Ave | PC | CC |
|---|---|---|---|---|---|---|---|---|---|---|---|---|---|
| | | PC | NC | dCq | dCq | PC | NC | dCq | dCq | PC | NC | dCq | dCq |
| | | EGFR Ex19 del | | | | EGFR T790M | | | | EGFR c719 | | | |
| Panel A | gBlock | 31.73 | 44.11 | 0.51 | 12.82 | 35.59 | 46.07 | 5.38 | 14.72 | 40.6 | 50 | 10.1 | 18.65 |
| | Horizon | 32.12 | | 0.14 | | 35.98 | | 5.6 | | 41.41 | | 10.43 | |
| | Standard | | | | | | | | | | | | |
| | | EGFR c861 | | | | EGFR cC797S | | | | KRAS c12 | | | |
| Panel B | gBlock | 35.78 | 41.53 | 4.16 | 10.41 | 29.79 | 42.67 | 0.83 | 11.6 | 35.85 | 40.59 | 5.23 | 9.87 |
| | Horizon | 36.63 | | 4.74 | | 30.75 | | 0.85 | | 34.96 | | 5.07 | |
| | Standard | | | | | | | | | | | | |
| | | EGFR Ex20ins ASV | | | | EGFR L858R | | | | PIK3CA c1047 | | | |
| Panel C | gBlock | 36.5 | 50 | 6.33 | 19.64 | 35.23 | 43.3 | 5.06 | 12.94 | 35.72 | 48.91 | 5.45 | 20.21 |
| | Horizon | 35.63 | | 6.97 | | 34.88 | | 5.22 | | 34.86 | | 5.2 | |
| | Standard | | | | | | | | | | | | |
| | | KRAS c61 | | | | PIK3CA c542 | | | | EGFR S768I | | | |
| Panel D | gBlock | 34.08 | 42.02 | 4.34 | 10.81 | 41 | 50 | 11.14 | 19.86 | 32.77 | 50 | 2.04 | 18.79 |
| | Horizon | 33.96 | | 3.84 | | | | | | 33.4 | | 3.29 | |
| | Standard | | | | | | | | | | | | |
| | | NRAS c12 | | | | NRAS c61 | | | | BRAF c600 | | | |
| Panel E | gBlock | 32.16 | 50 | 11.15 | 20.89 | 33.84 | 42.25 | 3.37 | 12.31 | 31.91 | 44.89 | 1.52 | 14 |
| | Horizon | 31.28 | | 10.94 | | 34.73 | | 3.15 | | | | | |
| | Standard | | | | | | | | | | | | |

It is clear that the Lung Cancer Multiplex Taqman assay is very effective in detecting the desired mutations. The assays detect their intended mutations in 15 targets in the reference mutant samples as well as synthetic gBlock mutant sequences.

The assays detect all targets at least 0.5% mutation in 5 ng total DNA input in the following 4 instruments: ABI QuantStudio 5, BioRad CFX384, Roche LC480II and ABI 7500 Fast Dx. The PCR run time is ˜3 hours. The assay uses a total of 5 tubes, each containing 3 targets and an internal control. The internal control is an amplicon of the ACTB gene.

The assays were optimized on and perform well on the Roche LightCycler® 480 II, Bio-Rad CFX384™ and ABI Quantstudio.

### Example 10

The Following is exemplary of XNA Oligomer Synthesis:

### Part I. Synthetic Procedure of the Fmoc Oxy-Aza-T XNA Monomer

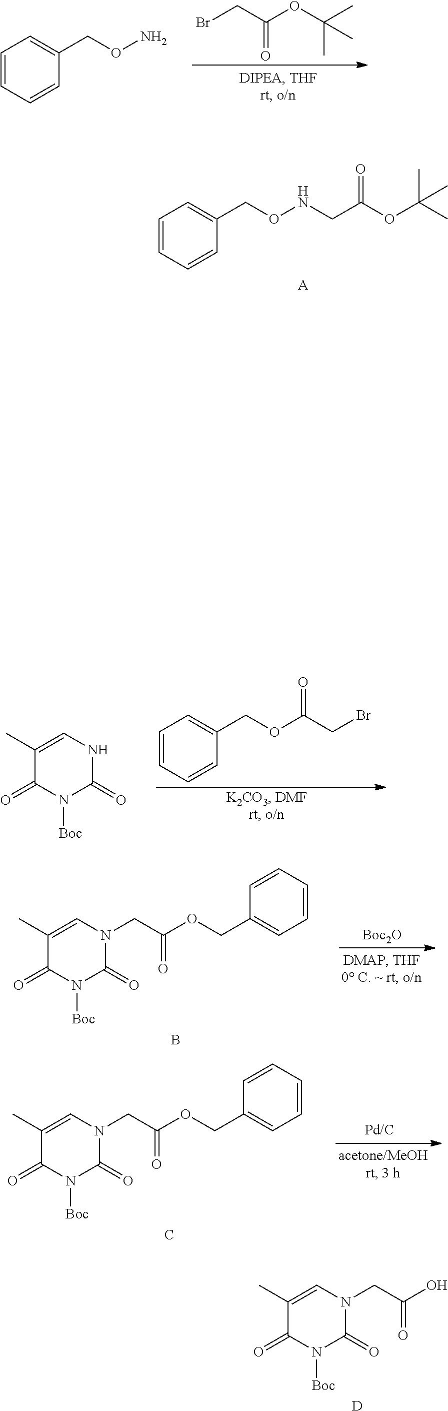

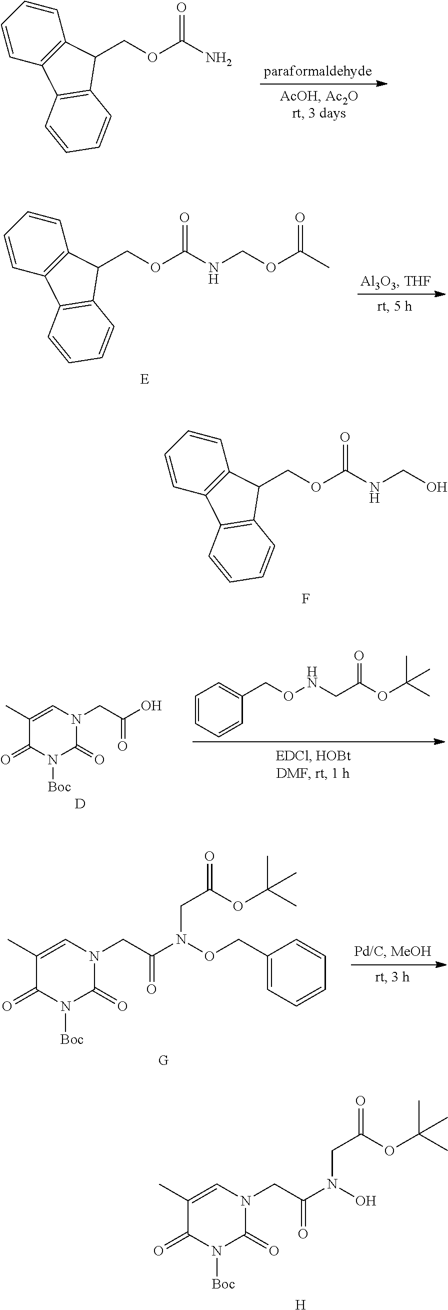

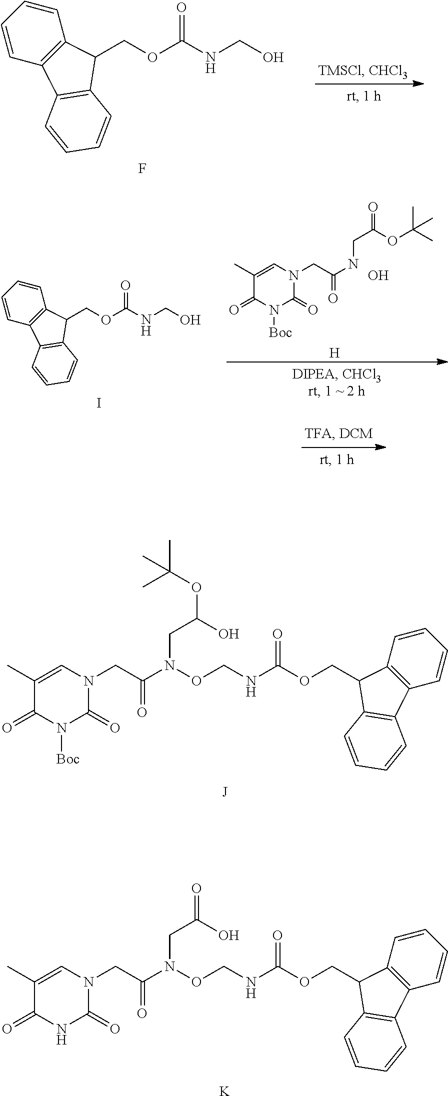

The other oxy-aza nucleotide Monomers A, C and G are prepared similarly with suitable protecting groups on the nucleoside bases.

Step 1: To a solution of O-benzylhydroxylamine (2.00 g, 15.9 mmol) and diisopropylethylamine (3.08 mL, 17.51 mmol) in THF (25 mL) was added dropwise tert-butyl 2-bromoacetate (2.5 mL, 16.71 mmol) in THF (10 mL). The reaction mixture was stirred at 50° C. for 4 hours then at room temperature overnight. Solvent was removed under vacuum to obtain crude which was purified by Biotage Isolera flash column to obtain title compound A (1.17 g, 29.4%) as a colorless oil.

Step 2: Thymine (3.00 g, 23.0 mmol) and potassium carbonate (3.30 g, 24.0 mmol) were dissolved in dry N,N-dimethylformamide (˜70 mL). Benzyl bromoacetate (3.50 mL, 22.0 mmol) was added dropwise and the reaction mixture was stirred at room temperature overnight. The suspension was filtered and solvent was removed to obtain a residue which was purified by Biotage flash column to obtain compound B (4.09 g, 61.4%) as a white solid.

Step 3: Benzyl 2-(5-methyl-2,4-dioxo-3,4-dihydropyrimidin-1(2H)-yl)acetate (3.00 g, 10.0 mmol), di-tert-butyl decarbonate (4.92 mL, 22.0 mmol), and 4-dimethylaminopyridine (2.56 g, 22 mmol) were added to THF (˜30 mL) at 0° C. The reaction mixture was stirred at 0° C. for 30 minutes and then at room temperature overnight. The solvent was removed. The residue was dissolved in dichloromethane (100 mL) and washed with water, brine, and dried over anhydrous MgSO₄, filtered and concentrated. The crude was purified by Biotage flash column to obtain compound C (2.91 g, 71.1%) as a white solid.

Step 4: To a solution of tert-butyl 3-(2-(benzyloxy)-2-oxoethyl)-5-methyl-2,6-dioxo-2,3-dihydropyrimidine-1(6H)-carboxylate (2.91 g, 7.38 mmol) in methanol (30 mL) and acetone (30 mL), 5% Pd/C (582 mg) was added. The reaction mixture was degassed with hydrogen 3 times and stirred at room temperature under hydrogen for 3 hours. The mixture was filtered with celite and washed with methanol and acetone. The filtrate was concentrated to obtain crude compound D (1.84 g, 83.3%).

Step 5: (9H-fluoren-9-yl)methyl carbamate (3.00 g, 12.0 mmol) and paraformaldehyde (0.43 g, 14.0 mmol), were suspended in a mixture of acetic acid (22.5 mL) and acetic anhydride (70 mL). The reaction mixture was stirred at room temperature for 3 days and then filtered. The solvent was removed by distillation in vacuum and the crude was purified by flash column to get compound E (3.46 g, 85.9%) as a white solid.

Step 6: ((((9H-fluoren-9-yl)methoxy)carbonyl)amino)methyl acetate (3.40 g, 10.0 mmol) was dissolved in THF (˜10 mL) and loaded on a 68-gram neutral alumina column. The loaded cartridge was allowed to stand for 5 hours then eluted by THF, and thereafter concentrated to obtain compound F (1.28 g, 43.5%) as a white solid.

Step 7: N,N-diisopropylethylamine (1.15 mL, 6.49 mmol) was added to a solution of 2-(3-(tert-butoxycarbonyl)-5-methyl-2,4-dioxo-3,4-dihydropyrimidin-1(2H)-yl)acetic acid (1.03 g, 3.245 mmol), tert-butyl 2-((benzyloxy)amino)acetate (0.89 g, 3.57 mmol), 1-Ethyl-3-(3-dimethylaminopropyl)carbodiimide-HCl (3.38 g, 17.13 mmol) and hydroxybenzotriazole hydrate (2.68 g, 17.13 mmol) in N,N-dimethylformamide (˜40 mL). The reaction mixture was stirred at room temperature overnight and diluted with dichloromethane (˜50 mL). The solution was washed with water, brine, dried over anhydrous MgSO₄, filtered and concentrated. The crude was purified by flash column to obtain compound G (1.08 g, 59.5%) as a white solid.

Step 8:To a solution of tert-butyl 3-(2-((benzyloxy)(2-(tert-butoxy)-2-oxoethyl)amino)-2-oxoethyl)-5-methyl-2,6-dioxo-2,3-dihydropyrimidine-1(6H)-carboxylate (Compound G; 1.08 g, 2.04 mmol) in methanol (10 mL), 5% Pd/C (216 mg) was added. The reaction mixture was degassed with hydrogen for 3 times and stirred at room temperature under hydrogen for 3 hours. The mixture was filtered by celite and washed with methanol. The filtrate was concentrated to obtain a crude compound H (865 mg, 97.6%) as white foam.

Steps 9 and 10: To a solution of (9H-fluoren-9-yl)methyl (hydroxymethyl)carbamate (Compound F; 1.03 g, 3.63 mmol) in chloroform (40 mL), trimethylsilyl chloride (0.93 mL, 7.267 mmol) was added dropwise and stirred at room temperature for 1 hour. After 1 hour, tert-butyl 3-(2-((2-(tert-butoxy)-2-oxoethyl)(hydroxy)amino)-2-oxoethyl)-5-methyl-2,6-dioxo-2,3-dihydropyrimidine-1(6H)-carboxylate (1.74 g, 4.00 mmol) and N,N-diisopropylethylamine (2.58 mL, 14.53 mmol) were added to the above solution. The reaction mixture was stirred at room temperature for 1 hour. The reaction mixture was washed with water, brine, dried over anhydrous Na₂SO₄, filtered, and concentrated to get the residue which was purified by flash column to get compound J (762 mg, 30.0%) as a white solid.

Step 11:To a solution of tert-butyl 3-(7-(2-(tert-butoxy)-2-oxoethyl)-1-(9H-fluoren-9-yl)-3,8-dioxo-2,6- dioxa-4,7-diazanonan-9-yl)-5-methyl-2,6-dioxo-2,3-dihydropyrimidine-1(6H)-carboxylate (0.60 g, 0.857 mmol) in dichloromethane (˜12 mL), trifluoroacetic acid was added (˜5 mL, 85.8 mmol) at 0˜5° C. The reaction mixture was stirred at room temperature for 1 hour. The mixture was concentrated to obtain a residue which was purified by Biotage Isolera flash column to obtain the title compound (220 mg, 48.0%) as an off-white solid.

¹H NMR (300 MHz, CDCl3): 10.3 (s, 1H), 8.75 (s, 1H), 7.88 (d, J=7.5 Hz, 2H), 7.69 (d, J=7.3 Hz, 2H), 7.44-7.29 (m, 5H), 4.92 (d, J=6.1 Hz, 2H), 4.66 (s, 2H), 4.40-4.37 (m, 2H), 4.25 (t, J=6.4 Hz, 1H), 4.08-3.97 (m, 2H), 1.73 (s, 3H) ppm.LC-MS [M+H]⁺: 508.97, [M+Na]⁺: 531.23. HPLC purity: 95.7% at 254 nm.

### Part II. Synthesis of Chemically-Modified EGFR c797S XNA, Using Fmoc Oxy-Aza-T XNA Monomer (Bold Red) to Replace the Regular Fmoc-T Monomer (Bold Black) as Specified Below

EGFR c797S
Regular-T original sequence:
SEQ ID NO: 69
5′-D-LYS-O-TTCGGCTGCCTCCTGG-3′
Partial Oxy-Aza-T Replacement Sequence:
SEQ ID NO: 70
5′-D-LYS-O-TTCGGCT_{OA}GCCT_{OA}CCTGG-3′
where OA is oxy-aza.

### a) Solid-Phase Synthesis Step

This step has been conducted on an INTAVIS MultiPep automatic synthesizer (INTAVIS Bioanalytical Instruments AG, Cologne, Germany), coupled with a compact Welch vacuum pump (4 m³ per hour ventilation rate), a 20-liter stainless steel waste container, and a long ventilation hose to discharge the solvent vapor and smell from the system into a nearby chemical fume hood.

In a typical 24-port (4×6) array plate, a micro column (0.5-ml capacity) with PTFE filters was inserted tightly into a chosen port. A certain weight of TentaGel resin (1 micromole, namely 10.0 mg resin at 0.10 mmol/gram loading capacity) was loaded to this column.

Four regular monomers (Fmoc-T/A/C/G) and O-linker monomer (Fmoc-AEEA-OH) were purchased commercially (98+% purity) and prepared freshly as 0.3 M solutions in N-methyl 2-pyrrolidone (NMP); Fmoc-D-Lysine(t-Boc) monomer as a 0.5 M solution in NMP. This unconventional Fmoc Oxy-Aza-T monomer was also made as a 0.3 M solution in a smaller 15-ml polypropylene vial (100 mg about 0.2 mmol dissolved in 600 uL of NMP solvent), and was accordingly given a new code of monomer in the program (perhaps like “oaT”?). All other reagents (from Sigma-Aldrich if not specified otherwise, with purity of 98% or higher) include 0.5 M DMF solution of HATU (from P3 BioSyetems Inc, 1-[Bis(dimethylamino)-methylene]-1H-1,2,3-triazolo[4,5-b]pyridinium 3-oxid hexafluorophosphate, Hexafluoro-phosphate Azabenzotriazole Tetramethyl Uronium) for carboxy activation, a base solution containing 1.2 M DIPEA and 1.8 M 2,6-lutidine (1:1, v/v) in DMF for acid scavenger, a 20% piperidine solution in DMF (v %) for Fmoc group deprotection, 5% (v %) acetic anhydride in DMF for amino capping procedure, NMP and methylene chloride and ethanol for column wash use.

After the preparative procedures above are completed, the XNA sequence was input to the operating PC's INTAVIS program with double check. The automatic synthesis on the TentaGel resin was started from the 3′ terminal of XNA sequence (namely from C-terminal) following this program, using a pre-set 1-micromole-scale double-coupling synthesis method. Briefly, in a typical cycle, a double deprotection, a double coupling and a single capping procedure was included to assure the sufficiently high-yielding and clean synthesis; a molar ratio of HATU/Base/monomer/amino=5:25:5:1 was chosen in general. The synthesizer was programmed to automatically repeat the cycles from 3′ end to 5′ end, till the 5′ end of the sequence that is the D-lysine terminus here. At this last cycle, the resin was thoroughly washed and then dried. Resin weight was found to increase obviously.

### b) Resin Cleavage and Side-Chain Deprotection

The dried resin was transferred to a 50-ml polypropylene centrifuge vial, using methylene chloride as the suspension medium for an easy and complete transfer, then dried in vacuum. A cocktail of TFA/m-cresol/TIPS/water (90:5:2.5:2.5, v %) was added (1000 uL for 1 umol resin), the cleavage/deprotection procedure was carried out at room temperature on an orbital shaker for 3 hrs at 160 cpm. The resin was then filtered out, the filtrate (˜1 mL) was mixed with 40-mL of cold anhydrous ether (0-5 Celsius degree), a significant amount of off-white loose precipitate appeared. The precipitate was collected and vacuum-dried after high-speed centrifuge (4500 cpm, 20 minutes) on a WAVERLY fixed-angle centrifuge. The crude solid was redissolved in about 300 ul of water for HPLC purification.

### c) HPLC Purification of Fmoc-ON XNA

Our Agilent HPLC 1100 system consists of a G1322A degasser, G1311A Quaternary Pump, G1313A automatic sampler, G1316A column compartment with temperature control and G1315B diode array detector.

A typical HPLC purification run is conducted as below on a Aeris peptide XB-C18 RP-HPLC column (100×4.5 mm, 3.6 um particle size): 5%-29% gradient of mobile phase B in 0-28 minutes (mobile phase A: 0.1% TFA in water; mobile phase B: 0.1% TFA in acetonitrile) for elation of the XNA product and byproduct peaks, followed by 29%- 60% wash for 4 minutes (28-32 min), and then 60%-5% wash back to equilibrate the column for the next run (32-36 min). Other parameters: 1.0 ml/min flow rate, column temperature 50.0+/−0.5 Celsius degree, UV detection at 260 nm and 205 nm simultaneously (detecting DNA base and TFA impurity respectively), a single sample injection as 100 ul each run.

The XNA product peak fractions (a main and sharp peak usually in the range of 17-23 min) were collected and combined, as the eluted solution of purified XNA (Fmoc-ON version).

### d) Lyophilization of Fmoc-ON XNA

The purified Fmoc-ON XNA solution (in mixed solvent of water and acetonitrile, with 0.1% TFA) was transferred to a 50-ml centrifuge vial (polypropylene) and frozen either in cold bath of dry-ice/acetone or −80 Celsius degree freezer, then subjected to lyophilization.

A 1200-ml LABCONCO flask including the frozen sample vial(s) was attached to a port of multifold of a LABCONCO desktop lyophilizer (Freezone 4.5 model) which was already stabilized at −40 Celsius degree and approximately 100 microbar (0.1 mmHg). The process continued usually for 8-48 hours depending on total sample volume. Upon completion of this process, a loose and white solid was obtained as the dried XNA product (Fmoc-ON version).

This version of purified XNA can be used directly after being re-dissolved in water or TE buffer. The product quantity can be calculated by the base concentration measured at 260 nm and the XNA solution total volume, then the synthetic yield (%) can be calculated. MALDI-TOF mass spectrum of the synthesized XNA (Fmoc-ON version) was measured on Shimadzu Axima MALDI-TOF mass spectrometer and data was recorded, using sinapinic acid as the matrix and the bovine cytochrome C protein as the molecular weight reference standard. If even higher water solubility is mandatory, then the deprotection of the terminal Fmoc group of the purified XNA above can be further processed, see Step (e) and Step (f) below.

### e) Additional D-Lysine Fmoc Deprotection and Further HPLC Purification

The purified XNA above is redissolved in small amount of DMF (e.g. 300 ul for each micromole), then a calculated amount of piperidine was added in at room temperature so as to make it a 10% piperidine/DMF solution, the deprotection only took a few minutes to complete. Following the deprotection, 40-ml of cold anhydrous ether is added to precipitate the crude product.

Another round of HPLC was repeated with the conditions listed above, the Fmoc-OFF XNA peak comes out earlier, usually in the range of 10-15 min window due to its increased hydrophilicity and thus less stronger adsorption on the RP-HPLC column. All product fractions were collected and combined.

### f) Further Lyophilization and Formulation

Lyophilization procedure is similar to the procedure (d) described above, during which the acetonitrile and TFA can be completely removed, leaving a final powder product of XNA (Fmoc-OFF version).

The product quantity can be calculated by the base concentration measured at 260 nm and the XNA solution total volume, and then the synthetic yield (%) can be calculated.

MALDI-TOF mass spectrum of the synthesized XNA (Fmoc-OFF version) was measured on Shimadzu Axima MALDI-TOF mass spectrometer and data was recorded, using sinapinic acid as the matrix and the bovine cytochrome C protein as the molecular weight reference standard.

The powder XNA is then redissolved in either pure water or TE buffer, as an aqueous solution of typically 200 micromolar concentration. The resulting solution can be either directly used for the subsequent XNA clamping-based qPCR or aliquoted (e.g. 50 ul=10 nmol) for lyophilization again to store for long term.

Other XNA oligomers can be synthesized in a similar fashion composed partially or entirely of oxy-aza, aza-aza and/or sulfa-aza (thio-aza) XNA monomers.

Other XNA sequences used in the invention and more in particular with respect to Example 6 of the invention includes:

EGFR G719
SEQ ID NO: 71
D-Lys-O-CG_{OA}GA_{AA}GCCC_{OA}AGCACTTTGAT
EGFR Ex19Del
SEQ ID NO: 72
D-Lys-O-C_{OA}G_{OA}G_{OA}A_{OA}G_{OA}ATGTTGCT_{OA}T_{OA}CTCTTAATTCC
EGFR T790
SEQ ID NO: 73
D-Lys-O-T_{AA}C_{AA}A_{AA}TCAC_{OA}GC_{OA}AGCTC
EGFR L858
SEQ ID NO: 74
D-Lys-O-GGCCAGC_{OA}C_{OA}CAAAAT_{AA}CTGT
NRAS G12
SEQ ID NO: 75
D-Lys-O-C_{OA}AA_{AO}CAC_{AA}C_{AA}AC_{OA}CTGCTCCAACCACCAC
NRAS A59
SEQ ID NO: 76
D-Lys-O-TTC_{OA}TTGTC_{OA}CA_{OA}GCT_{AA}GTAT_{AA}CCAGTATG
KRAS G12
SEQ ID NO: 77
D-Lys-O-C_{AA}TACGCCACC_{OA}AGCTC_{OA}CAACTACCA
KRAS A59
SEQ ID NO: 78
D-Lys-O-C_{OA}TCTTGACCT_{OA}GCT_{OA}GTGT_{AA}CGAG
KRAS A146
SEQ ID NO: 79
D-Lys-O-T_{OA}GTCTTT_{AA}GCTG_{OA}ATGT
APC E1309
SEQ ID NO: 80
D-Lys-O-C_{AA}TGAC_{OA}CTAGT_{OA}TCCAAT_{AA}CTTTTCTT
P1K3CA H1047
SEQ ID NO: 81
D-Lys-O-A_{OA}ATGAT_{AA}GCACATCAT_{OA}GGTGGCTG
CTNNB1 S45
SEQ ID NO: 82
D-Lys-O-C_{AA}TCCTT_{OA}CTCT_{AA}GAG_{OA}TG
BRAF V600
SEQ ID NO: 83
D-Lys-O-A_{OA}TC_{OA}GAGAT_{AA}TT_{OA}CACT_{AA}GTAGCTAGAC

In sequences 71 through 83 the subscripts designations OA and AA stand for oxy-aza and aza-aza moieties in the Xenonucleic acid.

All literature and similar materials cited in this application including, but not limited to, patents, patent applications, articles, books, treatises, and internet web pages, regardless of the format of such literature and similar materials, are expressly incorporated by reference in their entirety for any purpose as if they were entirely denoted. In the event that one or more of the incorporated literature and similar materials defines or uses a term in such a way that it contradicts that term's definition in this application, this application controls.

Although the foregoing description contains many specifics, these should not be construed as limiting the scope of the present invention, but merely as providing illustrations of some of the presently preferred embodiments. Similarly, other embodiments may be devised without departing from the spirit or scope of the present invention. Features from different embodiments may be employed in combination. The scope of the invention is, therefore, indicated and limited only by the appended claims and their legal equivalents rather than by the foregoing description. All additions, deletions and modifications to the invention as disclosed herein which fall within the meaning and scope of the claims are to be embraced thereby.

What is claimed is:

1. A multiplex method for enriching a plurality of target polynucleotide sequences containing genetic mutations/variations associated with lung cancer, said method comprising: (a) providing a biological sample; (b) isolating DNA from said biological sample; said DNA including said plurality of target polynucleotide sequences containing genetic variations associated with lung cancer; (c) providing a plurality of primer probes targeted to said plurality of target polynucleotide sequences said primer probes allowing formation of a PCR process product; (d) providing a plurality of target specific xenonucleic acid clamps oligomer probes specific for wildtype polynucleotide sequences; so that during the qPCR process only mutant templates are amplified: (e) admixing the plurality of primer probes and the plurality of xenonucleic clamping probes with the target nucleic acid sample; (f) performing a PCR amplification process in a reaction solution under hybridization conditions thereby generating multiple amplicons; and (g) detecting said amplicons and wherein said xenonucleic acid clamps have aza-aza, thio-aza and oxy-aza chemical functionality and selected from the group consisting of the following chemical structures: 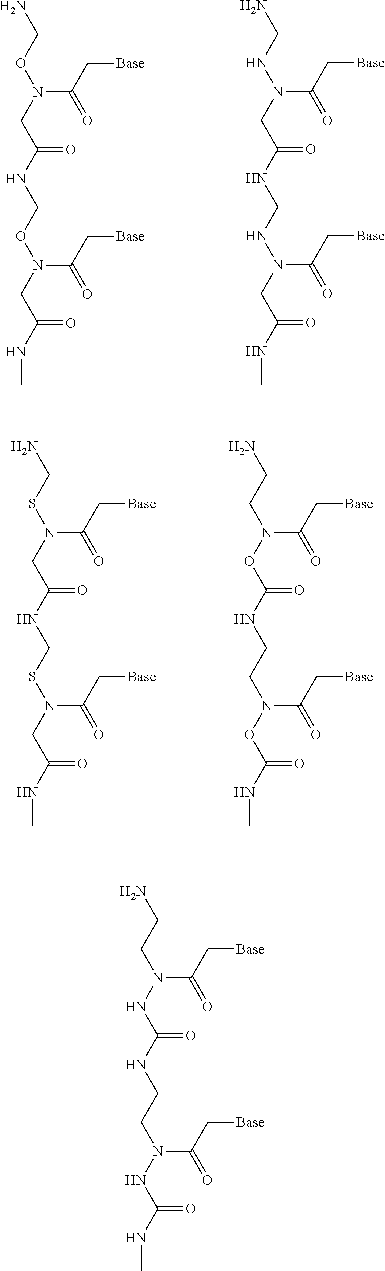
where base is selected from the group consisting of adenine, cytosine, guanine, thymine and uracil.

2. The method of claim 1, wherein said detection employs oligonucleotide probes specific for hybridization of variant polynucleotide amplicon sequences.

3. The method of claim 1, wherein the target polynucleotide sequences containing genetic mutations/variations are in a gene selected from the group consisting of: EGFR gene (codons 858, 746, 790, 861, 719, 768, and 769), KRAS gene (codons 12 and 61), NRAS gene (codons 12 and 61), PIK3CA (codons 542 and 1047), and BRAF (codon 600) associated with the non-small cell lung cancer (NSCLC).

4. A method for enriching a plurality of multiple target polynucleotide sequences containing a genetic mutations/variations said method comprising: (a) providing a biological sample; (b) isolating DNA from said biological sample; said DNA including said plurality of multiple target polynucleotide sequences containing genetic mutations/variations; (c) providing a library of amplifying primer probes targeted to said plurality of multiple target polynucleotide sequences containing a genetic mutations/variations; said primer probes allowing formation of PCR process products; (d) providing a library of target specific xenonucleic acid clamp oligomer probes specific for a plurality of multiple wildtype polynucleotide sequences so that during the qPCR process only mutant templates are amplified; (e) admixing the primer probes and the xenonucleic clamping probes with the multiple target nucleic acid samples; (f) performing a PCR amplification process in a reaction solution under hybridization conditions thereby generating multiple amplicons; and (g) detecting said amplicons and wherein said xenonucleic acid clamps have aza-aza, thio-aza and oxy-aza chemical functionality and selected from the group consisting of the following chemical structures; 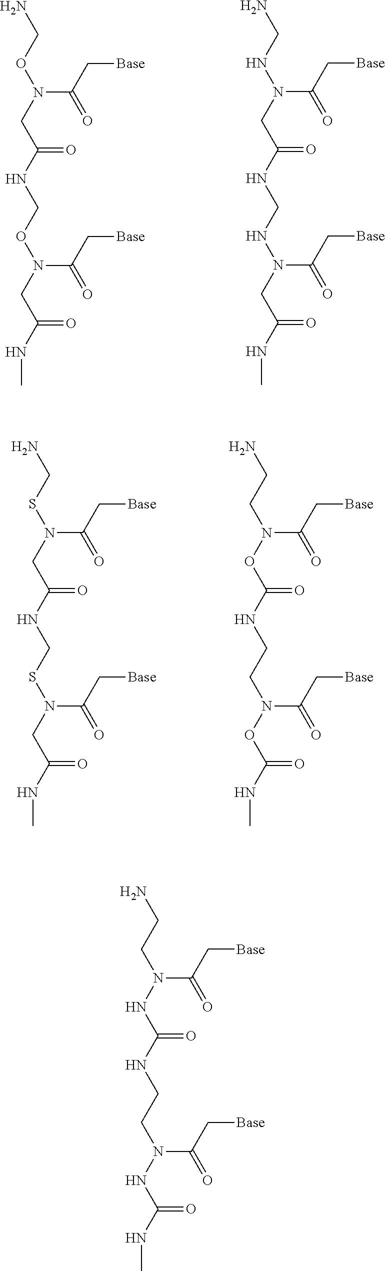
where base is selected from the group consisting of adenine, cytosine, guanine, thymine and uracil.

5. The method of claim 4, wherein the target polynucleotide sequences containing genetic mutations/variations are in a gene selected from the group consisting of: EGFR gene (codons 858, 746, 790, 861, 719, 768, and 769), KRAS gene (codons 12 and 61), NRAS gene (codons 12 and 61), PIK3CA (codons 542 and 1047), and BRAF (codon 600) associated with the non-small cell lung cancer (NSCLC).

6. The method of claim 4, wherein said target specific xenonucleic acid clamps provides a Tm differential of about 15°-20° C.

7. The method of claim 3, wherein said detection employs oligonucleotide probes specific for hybridization of variant polynucleotide amplicon sequences.

8. A multiplex method for conducting minimally invasive biopsies in a mammalian subjects suspected of a having lung cancer, said method comprising: (a) providing biological samples derived from said mammalian subjects; (b) isolating DNA from said biological samples; said DNA including a plurality of multiple target polynucleotide sequences containing genetic mutations/variations; (c) providing a library of amplifying primer probes targeted to said plurality of multiple target poly-nucleotide sequences containing genetic mutations/variations; said primer probes allowing formation of PCR process products; (d) providing a library of target specific xenonucleic acid clamp oligomer probes specific for a plurality of multiple wildtype polynucleotide sequences so that during the qPCR process only mutant templates are amplified; (e) admixing the primer probes and the xenonucleic clamping probes with the plurality of multiple target nucleic acid samples; (f) performing a PCR amplification process in a reaction solution under hybridization conditions thereby generating multiple amplicons; and (f) detecting said amplicons and wherein said xenonucleic acid clamps have aza-aza, thio-aza and oxy-aza chemical functionality and selected from the group consisting of the following chemical structures; 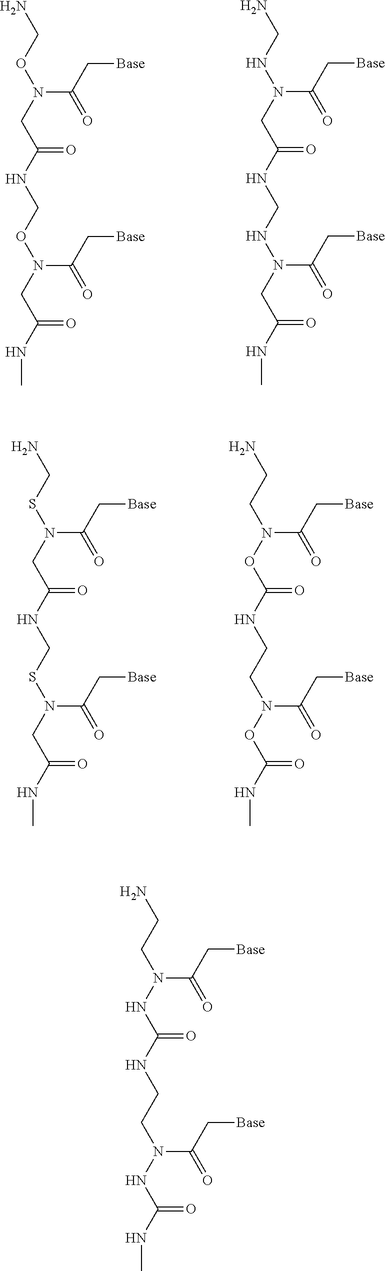
where base is selected from the group consisting of adenine, cytosine, guanine, thymine and uracil.

9. The method of claim 8, wherein the target polynucleotide sequences containing genetic mutations/variations are in a gene selected from the group consisting of: EGFR gene (codons 858, 746, 790, 861, 719, 768, and 769), KRAS gene (codons 12 and 61), NRAS gene (codons 12 and 61), PIK3CA (codons 542 and 1047), and BRAF (codon 600) associated with the non-small cell lung cancer (NSCLC).

10. The method of claim 8, wherein said target specific xenonucleic acid clamps provides a Tm differential of about 15° -20° C. during the qPCR process so only mutant templates are amplified

11. The method of claim 8, wherein said biological sample are cells derived from said mammalian subjects.

12. The method of claim 8, wherein said target polynucleotides containing genetic mutations/variations are derived from free circulating cell free polynucleotides derived from said mammalian subject.

13. The method of claim 8, which includes using multiple Xenonucleic acid clamp probes and amplifying primers targeted to multiple polynucleotide sequences.
